(19) United States
(12) Patent Application Publication
(10) Pub. No.: US 2017/0322216 A1
(43) Pub. Date: Nov. 9, 2017

(54) PANCREATIC CANCER DIAGNOSTIC

(71) Applicant: Fred Hutchinson Cancer Research Center, Seattle, WA (US)

(72) Inventors: Paul Lampe, Seattle, WA (US); Sunil R. Hingorani, Seattle, WA (US)

(21) Appl. No.: 15/512,763

(22) PCT Filed: Sep. 22, 2015
(86) PCT No.: PCT/US2015/051483

§ 371 Date: Mar. 20, 2017

Related U.S. Application Data
(60) Provisional application No. 62/054,883, filed on Sep. 24, 2014.

(51) Int. Cl. G01N 33/574

(52) U.S. Cl. CPC G01N 33/57438

(57) ABSTRACT

The present disclosure provides methods of using certain biomarker expression profiles in the detection, diagnosis, prognosis, or development of treatment regimens for various cellular hyperproliferative disorders of the pancreas. For example, methods comprise detecting whether the concentration of ERBB2, ESR1, and TNC in a test biological sample from a subject is elevated as compared to a control.

### CROSS REFERENCE TO RELATED APPLICATION

This application claims the benefit under 35 U.S.C. §119(e) to U.S. Provisional Application 62/054,883 filed Sep. 24, 2014, which application is incorporated by reference herein in its entirety.

### STATEMENT OF GOVERNMENT INTEREST

This invention was made with government support under Grant No. HHSN268200960003 awarded by National Heart, Lung, and Blood Institute of the National Institutes of Health, and Grant No. U01CA152746 awarded by the National Cancer Institute. The government has certain rights in the invention.

### BACKGROUND

Survival rates for many cancers including breast, colon and prostate have improved significantly in the past two decades, but the prognosis for pancreatic ductal adenocarcinoma (PDA), or pancreas cancer, has remained dismal. Five-year survival rates remained unchanged at ˜6% from 2002-2008, which is of additional concern given the 1.2% annual increase in incidence from 1999-2010. Surgical resection is the only curative option, but the majority of patients (>80%) present with unresectable disease at diagnosis, highlighting the need for improved early detection strategies (Klein et al., PLoS ONE 8:e72311, 2013). Patients diagnosed with localized, resectable disease have 5-year survival rates that improve from around 5% for advanced disease to a modest 20% (Ferrone et al., Surgery 152:S43-9, 2012), with a median post-resection survival of ˜17 months (Yeh et al., Expert Opin. Ther. Targets 11:673-94, 2007). These results reflect the micrometastatic capability of PDA early in disease progression and the challenges in detecting occult disseminated disease. Thus, tests that lead to earlier diagnosis are greatly needed to improve upon current survival rates.

The retroperitoneal location of the pancreas and its cargo of digestive enzymes further impede safe and efficient biopsy. The only FDA-approved blood-based biomarker for pancreatic cancer is CA19-9, but with sensitivities and specificities ranging from 60-70% and 70-85%, respectively (Goonetilleke et al., Eur. J. Surg. Oncol. 33:266-70, 2007), it is not recommended for use for screening, as a diagnostic, or to determine operability. Instead, CA19-9 is typically used to assess response to treatment and/or disease recurrence in people that express high levels at diagnosis (Winter et al., J. Surg. Oncol. 107:15-22, 2012 and Locker et al., J. Clin. Oncol. 24:5313-27, 2006).

Therefore, there remains a need for identifying pancreas cancer markers useful for disease detection or that are present and observable at, for example, preclinical stages. The present disclosure meets such needs, and further provides other related advantages.

### BRIEF DESCRIPTION OF THE DRAWINGS

FIGS. 1A-1D depict cross-species antibody microarray array results for murine KPC pre-invasive (A) and invasive (B) pancreas cancer plasma and for human pre-diagnostic plasma samples from the WHI (D). Plasma samples were drawn up to four years prior to death from PDA, and patients were diagnosed with all stages of disease, as determined using the T, N, M classification system and the patient information provided by WHI. The stage at diagnosis only influenced time from diagnosis to death in patients with stage IV disease (C). Statistical significance was computed using an unpaired 2-tailed t-test in GraphPad Prism 5.0. *p-value <0.05 for stage IA and IIA vs. IV; **p-value <0.01 for stage IB and IB vs. IV; ***p-value <0.001 for stage III vs IV. Samples from patients where stage at diagnosis could not be determined were not included in these comparisons. Volcano plots depict the p-values and accompanying odds ratios (OR) for each antibody feature on the array platform as determined using logistic regression analyses for the mouse arrays and a paired regression analyses for the human arrays. Candidate markers with p-values<0.05 are found above the red line (estimated) and the number of significant candidates—both up regulated and down regulated in disease versus control plasma—are indicated in the top left corner for each dataset.

FIGS. 2A-2H depict cross-species identification of elevated TNC and ERBB2 in KPC pancreas cancer plasma and human pre-diagnostic plasma and, for TNC, in diagnostic plasma samples. M-value plots for TNC and ERBB2 show elevated plasma levels in human pre-diagnostic (A and B) and mouse KPC plasma (D and E), respectively. TNC is also elevated in diagnostic human plasma (G) and KPC tumor tissue (F). Normalized M-values (red/green coefficients) for case and control samples are plotted along with the mean and standard deviations for each dataset. Paired 2-tailed t-tests were used to determine statistical significance for human pre-diagnostic plasma and unpaired 2-tailed t-tests for mouse plasma and tissue and human diagnostic plasma datasets. For the pre-diagnostic paired analyses (A), red dots in M-values plots=top ⅓ of M-values (ranked based on the difference between M-values for matched cases and controls [Mcase-Mcontrol]); blue dots=middle ⅓; green dots=bottom ⅓. (G) and (H) show human diagnostic data for TNC and CA19-9, respectively. All statistical analyses were conducted in GraphPad Prism 5.0. *p-value <0.05; **p-value <0.01.

FIG. 3 depicts immunohistochemistry (IHC) of TNC (Panels A-D) and accompanying serial sections stained with hematoxylin and eosin (Panels E-H) in murine tumor tissue, which show the emergence of TNC expression at early pre-invasive stages (PanIN-1; Panels A and E) that increases with progression to invasive PDA. IHC of TNC associated with a PanIN-3 and invasive adenocarcinoma are shown in Panels B and C, respectively. TNC deposition does not increase, however, in a model of chronic pancreatitis (Panel D). Scale bar, 10 μm.

FIGS. 4A-D depict receiver operator curve (ROC) analysis of prediagnostic and diagnostic samples. Specificity and sensitivity for a panel differentiating incipient PDA from controls in WHI pre-diagnostic or CATPAC diagnositc samples are plotted on x- and y-axes, respectively. (A) A 3-marker ESR1, ERBB2 and TNC panel for the pre-diagnostic plasma sample set. The combined area under the curve (AUC) for this panel=0.68 for the pre-diagnostic sample set. (B) A 3-marker ESR1, ERBB2 and TNC panel for the diagnostic plasma sample set (AUC=0.86). (C) A 4-marker ESR1, ERBB2, TNC, and CA19-9 panel for the pre-diagnostic plasma sample set (AUC=0.71). (D) A 4-marker ESR1, ERBB2, TNC, and CA19-9 panel for the diagnostic plasma sample set (AUC=0.97).

### DETAILED DESCRIPTION

The instant disclosure provides methods for detecting biomarkers associated with a risk for developing a pancreas hyperproliferative disorder (e.g., pancreatic ductal adenocarcinoma or a precursor lesion) and allows for the detection, diagnosis, prognosis, or development of treatment regimens of a pancreas hyperproliferative disorder. For example, the methods comprise detecting the concentration of at least one biomarker in a test biological sample from a subject and determining if the concentration of the biomarker in the test biological sample is elevated compared to a control. The concentration of the biomarker in the sample may be measured by detecting the amount of biomarker in the sample that specifically binds to a binding molecule (e.g., an antibody or antigen binding fragment thereof). The methods disclosed herein can utilize an antibody array or antibody sandwich assay platform (e.g., ELISA) that allows for the isolation and detection of biomarkers if present in a sample. The biomarkers found in a biological sample, such as plasma, can be captured by antibodies specific to the biomarker and detected directly via labeling of the proteins or by antibodies that comprise a reporter (e.g., a fluorescently or chromogenically labeled antibody). The biomarkers identified herein are significantly elevated in subjects that have a pancreas hyperproliferative disorder. Furthermore, these methods can be combined with other known diagnostic methods for the disease of interest to further increase the sensitivity of the detection, diagnosis, prognosis or development of treatment regimens.

Therefore, the present disclosure provides powerful diagnostic tools that can be utilized to determine the risk, diagnosis, or progression of a pancreas hyperproliferative disorder.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives or enumerated components. As used herein, the terms “include,” “have” and “comprise” are used synonymously, which terms and variants thereof are intended to be construed as non-limiting.

As used herein, “hyperproliferative disorder” refers to any of a number of diseases that are characterized by excessive or inappropriate cell division leading to pathological changes. Neoplasia is an example of such a condition whereby abnormal cell division and tissue growth occurs more rapidly than normal and continues after the stimuli that initiated the new growth ceases. Neoplasms show partial or complete lack of structural organization and functional coordination with normal tissue and usually form a distinct mass of tissue which can be either benign (benign tumor) or malignant (cancer). Malignant tumors can occur in virtually any tissue (e.g., pancreas, breast, prostate, colon, lung, skin) and are characterized by local invasion of tissue and distant metastasis often leading to death. Benign tumor growth is typically not metastatic or locally invasive, but can lead, in certain circumstances, to severe disease and even death due to altered tissue function or tumor growth compressing or damaging adjacent critical structures (e.g., arteries, veins, nerves).

A “pancreas hyperproliferative disorder” or “PHD” is a hyperproliferative disorder as described above that begins in tissues of the pancreas. The pancreas is a glandular organ in the digestive system and endocrine system of vertebrates. In humans, it is located in the abdominal cavity behind the stomach. Pancreas hyperproliferative disorders can include, for example, a precursor lesion, neoplastic lesion, carcinoma in situ, adenoma, or pancreatic ductal adenocarcinoma (PDA). Most cases of PDA begin as small, precursor lesions. A “precursor lesion” is a hyperproliferative disorder that has not invaded the surrounding tissue or metastasized. Non-limiting examples of precursor lesions include intraductal papillary mucinous neoplasms (IPMNs), mucinous cystic neoplasms (MCNs), and pancreatic intraepithelial neoplasia (PanIN). PanIN lesions can be further characterized as PanIN1, PanIN2, or PanIN3. Several precursor lesions of pancreatic cancer have been characterized and described in Hruban et al. (2007) Gastroenterol. Clin. North Am. 36:831-vi, herein incorporated by reference in its entirety.

As used herein, “prognosis” is the likelihood of the clinical outcome for a subject afflicted with a specific disease or disorder. With regard to cancer, the prognosis is a representation of the likelihood (probability) that the subject will survive (such as for 1, 2, 3, 4 or 5 years) and/or the likelihood that the tumor will metastasize. A “poor prognosis” indicates a greater than 50% chance that the subject will not survive to a specified time point (such as 1, 2, 3, 4 or 5 years), and/or a greater than 50% chance that the tumor will metastasize. In several examples, a poor prognosis indicates that there is a greater than 60%, 70%, 80%, or 90% chance that the subject will not survive and/or a greater than 60%, 70%, 80% or 90% chance that the tumor will metastasize. Conversely, a “good prognosis” indicates a greater than 50% chance that the subject will survive to a specified time point (such as 1, 2, 3, 4, or 5 years), and/or a greater than 50% chance that the tumor will not metastasize. In several examples, a good prognosis indicates that there is a greater than 60%, 70%, 80%, or 90% chance that the subject will survive and/or a greater than 60%, 70%, 80% or 90% chance that the tumor will not metastasize.

The methods disclosed herein are used to detect biomarkers that indicate the risk, diagnosis, progression, prognosis, or monitoring of a pancreas hyperproliferative disorder. “Biomarker” refers to a molecule, compound, or other chemical entity that is an indicator of a biological condition (e.g., disease or disorder). Exemplary biomarkers include proteins (e.g., antigens or antibodies), carbohydrates, cells, viruses, nucleic acids, or small organic molecules. For example, a biomarker may be a gene product that is (a) expressed at higher or lower levels, (b) present at higher or lower levels, (c) a variant or mutant of the gene product, or (d) simply present or absent, in a cell or tissue sample from a subject having or suspected of having a disease as compared to an undiseased tissue or cell sample from a subject having or suspected of having a disease, or as compared to a cell or tissue sample from a subject not having or suspected of having a disease. That is, one or more gene products are sufficiently specific to the test sample that one or more may be used to identify, predict, or detect the presence of disease, risk of disease, or provide information for a proper or improved therapeutic regimen. A biomarker may refer to two or more components (e.g., proteins, nucleic acids, carbohydrates, or a combination thereof) that bind together or associate non-covalently to form a complex.

The term “polypeptide” as used herein refers to a compound made up of amino acid residues that are linked by peptide bonds. The term “protein” may be synonymous with the term “polypeptide” or may refer, in addition, to a complex of two or more polypeptides. Generally, polypeptides and proteins are formed predominantly of naturally occurring amino acids.

A “binding domain” or “binding region,” as used herein, refers to a protein, polypeptide, oligopeptide, or peptide (e.g., antibody, receptor) that possesses the ability to specifically recognize and bind to a target (e.g., antigen, ligand). A binding domain includes any naturally occurring, synthetic, semi-synthetic, or recombinantly produced binding partner for a biological molecule or another target of interest. Exemplary binding domains include single chain antibody variable regions (e.g., domain antibodies, sFv, single chain Fv fragment (scFv), Fab, F(ab′)₂), receptor ectodomains, or ligands. A variety of assays are known for identifying binding domains of the present disclosure that specifically bind a particular target, including Western blot, ELISA, and Biacore® analysis.

The term “epitope” includes any protein determinant capable of specific binding to an immunoglobulin or receptor (e.g., T-cell receptor). Epitopic determinants usually consist of chemically active surface groupings of molecules, such as amino acids or sugar side chains, and usually have specific three dimensional structural characteristics, as well as specific charge characteristics.

Exemplary binding domains comprise immunoglobulin light and heavy chain variable domains (e.g., scFv, Fab) and are herein referred to as “immunoglobulin binding domains.” In certain embodiments, a binding domain is part of a larger polypeptide or protein and is referred to as a “binding protein.” An “immunoglobulin binding protein” refers to a polypeptide containing one or more immunoglobulin binding domains, wherein the polypeptide may be in the form of any of a variety of immunoglobulin-related protein scaffolds or structures, such as an antibody or an antigen binding fragment thereof, a scFv-Fc fusion protein, or a fusion protein comprising two or more of such immunoglobulin binding domains or other binding domains.

Sources of binding domains include antibody variable regions from various species (which can be formatted as antibodies, sFvs, scFvs, Fabs, or soluble V_{H} domain or domain antibodies), including human, rodent, avian, leporine, and ovine. Additional sources of binding domains include variable regions of antibodies from other species, such as camelid (from camels, dromedaries, or llamas; Ghahroudi et al., FEBS Letters 414:521, 1997; Vincke et al., J. Biol. Chem. 284:3273, 2009; Hamers-Casterman et al., Nature, 363:446, 1993 and Nguyen et al., J. Mol. Biol., 275:413, 1998), nurse sharks (Roux et al., Proc. Nat'l. Acad. Sci. (USA) 95:11804, 1998), spotted ratfish (Nguyen et al., Immunogenetics, 54:39, 2002), or lamprey (Herrin et al., Proc. Nat'l. Acad. Sci. (USA) 105:2040, 2008 and Alder et al., Nature Immunol. 9:319, 2008). These antibodies can apparently form antigen-binding regions using only heavy chain variable region, i.e., these functional antibodies are homodimers of heavy chains only (referred to as “heavy chain antibodies”) (Jespers et al., Nature Biotechnol. 22:1161, 2004; Cortez-Retamozo et al., Cancer Res. 64:2853, 2004; Baral et al. Nature Med. 12:580, 2006, and Barthelemy et al. J. Biol. Chem. 283:3639, 2008).

An alternative source of binding domains for use with the methods of this disclosure includes ligand(s), extracellular domains of receptors, sequences that encode random peptide libraries or sequences that encode an engineered diversity of amino acids in loop regions of alternative non-antibody scaffolds, such as fibrinogen domains (see, e.g., Weisel et al., Science 230:1388, 1985), Kunitz domains (see, e.g., U.S. Pat. No. 6,423,498), ankyrin repeat proteins (Binz et al., J. Mol. Biol. 332:489, 2003 and Binz et al., Nature Biotechnol. 22:575, 2004), fibronectin binding domains (Richards et al., J. Mol. Biol. 326:1475, 2003; Parker et al., Protein Eng. Des. Select. 18:435, 2005 and Hackel et al., J. Mol. Biol. 381:1238, 2008), cysteine-knot miniproteins (Vita et al., Proc. Nat'l. Acad. Sci. (USA) 92:6404, 1995; Martin et al., Nature Biotechnol. 21:71, 2002 and Huang et al. Structure 13:755, 2005), tetratricopeptide repeat domains (Main et al. Structure 11:497, 2003 and Cortajarena et al., ACS Chemical Biology 3:161, 2008), leucine-rich repeat domains (Stumpp et al. J. Mol. Biol. 332:471, 2003), lipocalin domains (see, e.g., WO 2006/095164, Beste et al. Proc. Nat'l. Acad. Sci. (USA) 96:1898, 1999 and Schonfeld et al., Proc. Nat'l. Acad. Sci. (USA) 106:8198, 2009), V-like domains (see, e.g., US Patent Application Publication No. 2007/0065431), C-type lectin domains (Zelensky and Gready, FEBS J. 272:6179, 2005; Beavil et al., Proc. Nat'l. Acad. Sci. (USA) 89:753, 1992 and Sato et al., Proc. Nat'l. Acad. Sci. (USA) 100:7779, 2003), mAb² or Fcab™ (see, e.g., PCT Patent Application Publication Nos. WO 2007/098934; WO 2006/072620), or the like (Nord et al., Protein Eng. 8:601, 1995; Nord et al., Nature Biotechnol. 15:772-777, 1997; Nord et al., European J. Biochem. 268:4269, 2001 and Binz et al., Nature Biotechnol. 23:1257, 2005).

Binding domains of this disclosure can be generated as described herein or by a variety of methods known in the art (see, e.g., U.S. Pat. Nos. 6,291,161 and 6,291,158). For example, binding domains or binding proteins of this disclosure may be identified by cloning the appropriate sequence of a ligand or of a receptor extracellular domain, or by screening a Fab phage library for Fab fragments that specifically bind to a target of interest (see Hoet et al., Nature Biotechnol. 23:344, 2005). Additionally, traditional strategies for hybridoma development using a target of interest as an immunogen in convenient systems (e.g., mice, HuMAb Mouse®, TC Mouse™, KM-Mouse®, llamas, chicken, rats, hamsters, rabbits, etc.) can be used to develop antibodies, binding domains or binding proteins of this disclosure.

A binding domain and a fusion protein thereof “specifically binds” a target if it binds the target with an affinity or Kₐ (i.e., an equilibrium association constant of a particular binding interaction with units of 1/M) equal to or greater than 10⁵ M⁻¹, while not significantly binding other components present in a test sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Kₐ of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Kₐ of up to 10⁸ M⁻¹, up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al., Ann. N.Y. Acad. Sci. 51:660, 1949; and U.S. Pat. Nos. 5,283,173, 5,468,614, or the equivalent).

Terms understood by those in the art of antibody technology are each given the meaning acquired in the art, unless expressly defined differently herein. The term “antibody” refers to an intact antibody comprising at least two heavy (H) chains and two light (L) chains inter-connected by disulfide bonds, as well as an antigen-binding portion of an intact antibody that has or retains the capacity to bind a target molecule. A monoclonal antibody or antigen-binding portion thereof may be non-human, chimeric, humanized, or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

The term “biological sample” includes a blood sample, biopsy specimen, tissue explant, organ culture, biological fluid or specimen (e.g., blood, serum, plasma, ascites, mucosa, lung sputum, saliva, feces, cerebrospinal fluid (CSF)) or any other tissue or cell or other preparation from a subject or a biological source. A “subject” or “biological source” may be, for example, a human or non-human animal, a primary cell or cell culture or culture adapted cell line including genetically engineered cell lines that may contain chromosomally integrated or episomal recombinant nucleic acid molecules, somatic cell hybrid cell lines, immortalized or immortalizable cell or cell lines, differentiated or differentiatable cells or cell lines, transformed cells or cell lines, or the like. In a preferred embodiment, a biological sample is from a human. By “human patient” is intended a human subject who is afflicted with, at risk of developing or relapsing with, any disease or condition associated with pancreas hyperproliferative disorder.

A biological sample is referred to as a “test sample” when being tested or compared to a “control.” A “control,” as used herein, refers to an undiseased sample from the same patient and same tissue, a sample from a subject not having or suspected of having the disease of interest, a pool of samples (e.g., including samples from two to about 100,000 subjects) from various subjects not having or suspected of having the disease of interest, or data from one or more subjects not having or suspected of having the disease of interest (e.g., a database containing information on biomarker levels from one to about 5,000 to about 10,000 to about 100,000 to about 1,000,000 or more subjects). In certain embodiments, a “test sample” is analyzed and the results (i.e., biomarker levels) compared to a “control” comprising an average or certain identified baseline level calculated from a database having data derived from a plurality of analyzed undiseased or normal samples.

A “reference” or “standard” may optionally be included in an assay, which provides a measure of a standard or known baseline level of a target molecule (e.g., “normal” level). In certain embodiments, a reference sample is a pool of samples (e.g., 2, 3, 4, 5, 6, 7, 8, 9, 10, or more samples combined) from healthy individuals (i.e., not having or suspected of having the disease of interest). In certain instances, a “test sample” and a “control sample” will be examined in an assay of the instant disclosure along with a reference sample. In these instances, the “test” and “control” samples may be collectively referred to as the “target samples” since they are being compared to a reference sample.

When referring to the level of the one or more biomarker in a test sample, “elevated” compared to a control, as used herein, means a statistically significant increase in level. In certain embodiments, the level of biomarker(s) in a test sample is elevated compared to a control in a statistically significant manner. In further embodiments, the level of biomarker(s) in a test sample is increased in a statistically significant manner. For example, the difference between test and control levels may be about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 15-fold, about 20-fold, about 30-fold, or more. In certain instances, a statistically significant difference includes when a biomarker is present in a test sample but is absent or undetectable in the control.

As used herein, “decreased” means the level of the one or more biomarkers of interests in a test sample is decreased in a statistically significant manner. In certain embodiments, the level of biomarker(s) in a test sample is decreased compared to a control in a statistically significant manner. For example, the difference between test and control levels may be about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 15-fold, about 20-fold, about 30-fold, or more. In certain instances, a statistically significant decrease includes when a biomarker is present in a control sample but is minimally present or detectable, or absent or undetectable in a test sample.

In certain embodiments of this disclosure, a subject or biological source may be suspected of having or being at risk for having a disease, disorder or condition, including a malignant, disease, disorder or condition. In certain embodiments, a subject or biological source may be suspected of having or being at risk for having a pancreas hyperproliferative disorder (e.g., pancreatic cancer), and in certain other embodiments of this disclosure the subject or biological source may be known to be free of a risk or presence of such disease, disorder, or condition.

As used herein, “risk” is the likelihood (probability) of a subject developing a pancreas hyperproliferative disorder. Risk is a representation of the likelihood that subject will develop a pancreas hyperproliferative disorder within a period of time (such as 1, 2, 3, 4 or 5 years). A “high risk” indicates a greater than 50% chance that the subject will develop a pancreas hyperproliferative disorder. In several examples, a high risk indicates that there is a greater than 60%, 70%, 80%, or 90% chance that a subject will develop a pancreas hyperproliferative disorder. Conversely, a “low risk” indicates a less than 50% chance that the subject will develop a pancreas hyperproliferative disorder. In several examples, a low risk indicates that there is a less than 10%, 20%, 30%, or 40% chance of developing a pancreas hyperproliferative disorder.

As used herein, “pre-diagnosis detection” refers to the detection of biomarkers prior to diagnosis of a pancreas hyperproliferative disorder by other methods known in the art. Examples of such methods used to diagnose a pancreas hyperproliferative disease include biopsy, endoscopic ultrasound, endoscopic retrograde cholangiopancreatography (ERCP), computed tomography, magnetic resonance imaging (MRI), or any combination thereof.

The term “array” refers to an arrangement of a plurality of addressable locations or “addresses” on a device or substrate. The locations can be arranged in two-dimensional arrays, three-dimensional arrays, or other matrix formats. The number of locations may range from two to several (e.g., 3, 4, 5, 10, 15, 20, 50, 100) to at least hundreds of thousands. Most importantly, each location represents a totally independent reaction site. A “binding protein array” refers to an array containing binding proteins, such as antibodies or other molecules containing a binding domain. An “address” on an array (e.g., a microarray) refers to a location at which a feature or element, for example, an antibody, is attached to the solid surface of the array. An array may be in any form, such as a microarray, an ELISA or a multiplex assay (e.g., xMAP® of Luminex®).

As used herein, the term “isolated” means that the molecule referred to is removed from its original environment, such as being separated from some or all of the co-existing materials in a natural environment (e.g., a natural environment may be a cell).

Methods to measure protein/polypeptide expression levels of selected biomarkers in the present disclosure include, but are not limited to: Western blot, immunoblot, sandwich assay (e.g., enzyme-linked immunosorbant assay (ELISA), array format), multiplex format (e.g., xMAP® from Luminex®), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF), mass spectrometry, microcytometry, microarray, microscopy, fluorescence activated cell sorting (FACS), flow cytometry, and assays based on a property of the protein including but not limited to DNA binding, ligand binding, or interaction with other protein partners. These methods can be used to detect statistically significant difference in biomarker levels between control and test samples.

In certain embodiments, provided herein are methods for detecting the risk of a pancreas hyperproliferative disorder by identifying the risk of the pancreas hyperproliferative disorder in a human subject when a test sample from the human subject has at least one biomarker antigen that is elevated compared to a control. The level of biomarker antigen in the sample is measured by detecting the amount of biomarker antigen in the sample that specifically binds to an antigen binding domain. The biomarker antigen is at least one of an ERBB2 antigen, an ESR1 antigen, a TNC antigen, or any combination thereof. In some embodiments, the risk of a pancreatic ductal adenocarcinoma (PDA) is identified. In other embodiments, the risk of a precursor lesion is identified. In certain embodiments, the need for further screening is identified.

ERBB2 is a receptor tyrosine-protein kinase, also known as HER2, HER2/neu, and CD340. As referred to herein, “ERBB2” refers to the human polypeptide represented by any one of or combination of the reference amino acid sequences of UniProtKB Nos. P04626-1, P04626-2, P04626-3, P04626-4, P04626-5, P04626-6, or a variant or fragment thereof. Therefore, while full-length ERBB2 can be detected in the methods disclosed herein, variants and fragments thereof also can be detected. An ERBB2 antigen comprises at least a fragment or variant of ERBB2 that is recognized by an ERBB2 binding molecule, such as an anti-ERBB2 antibody (e.g., ABM Ab-1248).

ESR1 is a nuclear hormone receptor, also referred to as estrogen receptor, ESR, and NR3A1. As referred to herein, “ESR1” refers to the human polypeptide represented by any one of or combination of the reference amino acid sequences of UniProtKB Nos. P03372-1, P03372-2, P03372-3, P03372-4, or a variant or fragment thereof. Therefore, while full-length ESR1 can be detected in the methods disclosed herein, variants and fragments thereof also can be detected. An ESR1 antigen comprises at least a fragment or variant of ESR1 that is recognized by an ESR1 binding molecule, such as an anti-ESR1 antibody (e.g., Santa Cruz sc-543; ABM Ab-118).

TNC is an extracellular matrix protein, referred to as Tenascin and HXB. As referred to herein, “TNC” refers to the human polypeptide represented by any one of or a combination of the reference amino acid sequences of UniProtKB Nos. P24821-1, P24821-2, P24821-3, P24821-4, P24821-5, P24821-6, or a variant or fragment thereof. Therefore, while full-length TNC can be detected in the methods disclosed herein, variants and fragments thereof also can be detected. A TNC antigen comprises at least a fragment or variant of TNC that is recognized by a TNC binding molecule, such as an anti-TNC antibody (e.g., SDI 4166.00.02).

As used herein, “variant” means a polypeptide having a substantially similar amino acid sequence to a reference sequence. For molecules such as proteins, a variant can include an addition or deletion of one or more amino acids at one or more internal sites in the amino acid sequence of the reference enzyme and/or substitution of one or more amino acid residues at one or more sites in the amino acid sequence of the reference enzyme. The variant can result from, for example, a genetic polymorphism or human manipulation. A variant of the reference polypeptide can have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the reference sequence as determined by sequence alignment programs and parameters known in the art.

As used herein, a “fragment” means a polypeptide that is lacking one or more amino acids that are found in the reference sequence. A fragment can comprise an antigen or epitope found in the reference sequence. A fragment of the reference polypeptide can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of amino acids of the amino acid sequence of the reference sequence.

In certain embodiments, provided herein are methods for diagnosing a pancreas hyperproliferative disorder by diagnosing the pancreas hyperproliferative disorder in a human subject when a test sample from the human subject has at least one biomarker antigen that is elevated compared to a control. The level of biomarker antigen in the sample is measured by detecting the amount of biomarker antigen in the sample that specifically binds to an antigen binding domain. The biomarker antigen is at least one of an ERBB2 antigen, an ESR1 antigen, a TNC antigen, or any combination thereof. In some embodiments, pancreatic ductal adenocarcinoma (PDA) is diagnosed. In other embodiments, a precursor lesion is diagnosed. In certain embodiments, the need for further screening is identified.

In certain embodiments, provided herein are methods for identifying a human subject in need of additional screening for a pancreas hyperproliferative disorder by identifying the human subject when a test sample from the human subject has at least one biomarker antigen that is elevated compared to a control. The level of biomarker antigen in the sample is measured by detecting the amount of biomarker antigen in the sample that specifically binds to an antigen binding domain. The biomarker antigen is at least one of an ERBB2 antigen, an ESR1 antigen, a TNC antigen, or any combination thereof. The additional screening comprises at least one of an endoscopic ultrasound with or without a fine needle aspirate biopsy, endoscopic retrograde cholangiopancreatography (ERCP), computed tomography, magnetic resonance imaging (MRI), and biopsy, or any combination thereof. In some embodiments, the pancreas hyperproliferative disorder is pancreatic ductal adenocarcinoma. In other embodiments, the pancreas hyperproliferative disorder is a precursor lesion.

In certain embodiments, provided herein are methods for monitoring progression, residual disease, or recurrence of a pancreas hyperproliferative disorder by detecting at least one biomarker antigen in a sample from a human subject that has received at least one treatment for the pancreas hyperproliferative disorder and comparing the expression of the biomarker antigen to a control. The level of biomarker antigen in the sample is measured by detecting the amount of biomarker antigen in the sample that specifically binds to an antigen binding domain. The biomarker antigen is at least one of an ERBB2 antigen, an ESR1 antigen, a TNC antigen, or any combination thereof. In some embodiments, a decrease in at least one of ERBB2, ESR1, and TNC indicates a reduction in tumor burden or a remission. In other embodiments, an increase in at least one of ERBB2, ESR1, and TNC indicates an increase in tumor burden or a recurrence of the pancreas hyperproliferative disorder. In some of these embodiments, the pancreas hyperproliferative disorder is pancreatic ductal adenocarcinoma (PDA). In other embodiments, the pancreas hyperproliferative disorder is a precursor lesion.

Cancer progression is characterized by stages. Staging is usually based on the size of the tumor, whether lymph nodes contain cancer cells, and whether the cancer has spread from the original site to other parts of the body. Stages of pancreatic cancer include stage 0, stage I, stage II, stage III and stage IV. In some embodiments, the pancreatic cancer is from any stage.

As used herein “residual disease” is any tumor cells that remain in a patient after a treatment or therapy. Examples of a therapy are described elsewhere herein. Tumor cells include malignant cells, neoplasia, dysplasia, and metastatic cells.

As used herein, “recurrence” is defined as the return of cancer after treatment and after a period of time during which the cancer cannot be detected. “Recurrent pancreatic cancer” is a pancreatic cancer that has come back after it has been treated. The cancer may come back in the pancreas or adjoining structures or organs, such as lymph nodes, portal vein, ligament of Treitz, celiac plexus, or superior mesenteric blood vessels; or other parts of the body including, for example, liver, lungs, adrenal glands, diaphragm or peritoneum.

In certain embodiments, provided herein are methods of evaluating the efficacy of a pancreas hyperproliferative disorder therapy in a human subject by administering a pancreas hyperproliferative disorder therapy to a human subject and determining the efficacy of the therapy. The efficacy is assessed by measuring the level of at least one biomarker antigen compared to a control. The level of biomarker antigen in the sample is measured by detecting the amount of biomarker antigen in the sample that specifically binds to an antigen binding domain. The biomarker antigen is at least one of an ERBB2 antigen, an ESR1 antigen, a TNC antigen, or any combination thereof. In some embodiments, the biomarker antigen includes an ERBB2 antigen, an ESR1 antigen, a TNC antigen, and a CA19.9 antigen. In some aspects of the method, the therapy is surgery, chemotherapy, cytotoxic therapy, immune mediated therapy, targeted therapies, radiation therapy, chemoradiotherapy, or any combination thereof. In some embodiments, the pancreas hyperproliferative disorder is a pancreatic ductal adenocarcinoma (PDA). In other embodiments, the pancreas hyperproliferative disorder is a precursor lesion.

“Efficacy” is a measure of how well a therapy treats or reduces disease burden, such as tumor size or number. A reduction in biomarker antigen levels is an indication of reduction in disease burden and good efficacy. No change in biomarker antigen levels or a reduced rate of increase in biomarker antigen levels can be an indication that a therapy is tumorostatic. No effect on a statistically significant rate of increase in biomarker antigen levels is an indication of poor efficacy, minimal efficacy or a lack of efficacy. In certain embodiments, efficacy can be correlated with survival time. For example, therapy that increases survival time in patients in a statistically significant manner as compared to a control is correlated with higher efficacy. Conversely, a therapy that does not increase survival time in a statistically significant manner as compared to control is correlated with poor, minimal or no efficacy.

In some embodiments, the methods described herein detect at least two or all three of the biomarker antigens of ERBB2, ESR1, and TNC. Accordingly, the at least two biomarker antigens can be selected from ERBB2/ESR1, ERBB2/TNC, ESR1/TNC, ERBB2/ESR1/TNC, or any combination thereof. In certain embodiments, any of the methods described herein further include detecting the level of a CA19-9 antigen. The biomarkers can be detected simultaneously or sequentially.

The CA19-9 antigen, also referred to as carbohydrate antigen 19-9, cancer antigen 19-9, or sialyl-Lewis A antigen, is an art recognized biomarker for monitoring pancreatic cancer. However, the use of CA19-9 as a screening test for pancreatic cancer has been discouraged due to a high level of false negatives and false positives (Duffy et al. (2010) Ann Oncol. 21:441-447).

In certain embodiments, described herein are methods for detecting the risk, diagnosis, progression, prognosis, or monitoring of a pancreas hyperproliferative disorder in a human subject comprise detecting the risk, diagnosis, progression, prognosis, or monitoring of a pancreas hyperproliferative disorder when a test sample from the human subject has at least a TNC antigen and a CA19-9 antigen that is elevated compared to a control. The level of TNC antigen and CA19-9 antigen in the sample is measured by detecting the amount of TNC antigen and CA19-9 antigen in the sample that specifically binds to an antigen binding domain. The method can further include detecting at least one of an ERBB2 antigen, an ESR1 antigen, or any combination thereof. In some embodiments, the pancreas hyperproliferative disorder is a pancreatic ductal adenocarcinoma (PDA). In other embodiments, the pancreas hyperproliferative disorder is a precursor lesion. In certain embodiments, the need for further screening is identified.

In certain embodiments, any of the methods disclosed herein can detect additional biomarkers of interest, such as the biomarkers listed herein in Table 1, Table 2, Table 3 or any combination thereof. More specifically, any of the methods disclosed herein can detect additional biomarkers of interest, such as, SEPT5, IL2RA, KRT16, GATA3, TLX3, CDK2AP1, STAT3, CLU, SERPINH1, HOXD13, BCL2, IL1A, MLLT10, DDB2, CD20, BRAF, STEAP2, PKM2, NDRG1, or any combination thereof.

In certain embodiments, any of the methods disclosed herein can detect additional biomarkers of interest that are decreased in a sample, such as the biomarkers listed herein in Table 1, Table 2, Table 3 or any combination thereof. Exemplary biomarkers that show a decrease as compared to a control include DPP10, ALDH1A1, AAMP, PCTK1, BCL3, EIF2C2, ITGB1, MAP2K3, GADD45G, FLT3, NPTX2, THSD1, N-PAC (GLYR1), TYK2, ICAM1, PLEK, TBC1D3, GPR125, ITGB1, COPB2, SPINT2, IL16, PAK2, LCP1, HIF1A, IGFBP2, CTSE, STAT3, EGFR, APOL1, AR, UBE2S, PRDX3, PEBP1, ABL1, BIRC5, MFI2, STAT6, XRCC3, CHEK1, ZNF331, HDAC2, ATP8B1, RAB5A, BRCA1, PTPN11, FN1, FCRL5, ELP2, KIF16B, PRDX3, NANOG, CD44, ALDH1A1, TGFB2, ANKRD30A, ORM1, TMEM173, CHEK1, KRT14, MUTYH, CLTC, DCLK3, PEBP1, ARMCX1, ANXA2, CREB1, VIL2 (EZR), NFKB1, SPINK1, PTEN, PLCG1, ADAM28, YWHAZ, WDR1, IRS1, MAPK3/MAPK1 (pThr202/Tyr204), STAT3, RPS5, LASP1, FKBP4, PRKAR2A, TIMP1, DPYSL3, CLU, THBS2, LASP1, ABL1, IL1B, IL5, MT3, EPRS, TPI1, KLK5, REG4, NTNG1, RAD54L, AFP, RELA, ALPPL2, BAD, SPINT2, BCR, FABP3, SMAD2, MET, PTPRF, POLE4, EIF4E, CCNA2, HAPLN1, or any combination thereof.

In some embodiments, any of the methods provided herein further include the detection of autoantibodies that are bound to or complexed with their natural autoantigens, referred to herein a autoantibody-autoantigen complexes.

Autoantibody-autoantigen complexes are identified, for example, using a high-density antibody microarray platform comprising approximately 3,600 antibodies printed in triplicate to detect autoantibody-autoantigen (autoAb-autoAg) complexes. Some of the microarray antibodies target the same proteins but are specific for different epitopes or peptide sequences. AutoAb-autoAg complexes found in a biological sample, such as serum or plasma, are captured on the array (e.g., preferably the autoantibody and the array antibody do not sterically hinder each other) and detected by a fluorescently labeled anti-antibody (e.g., an anti-human immunoglobulin G (IgG)). Although autoAg found free in plasma (i.e., not complexed to autoantibody) and other proteins might bind at a specific spot on the array, no signal will be detected since the labeled anti-antibodies will not bind to the molecules at that spot. In addition, free autoantibodies (i.e., not bound to antigen) will be washed away from the array surface since the array will only bind antigen, not antibodies. Thus, the methods provided herein will detect autoAb-autoAg complexes present in a sample and represented on the antibody microarray.

While not wishing to be bound by theory, autoantibodies are potential biomarkers for disease since immune surveillance often occurs early during a disease process and antibodies can detect a disease before overt symptoms occur (particularly in chronic diseases) (Anderson and LaBaer, J. Proteome Res. 4:1123, 2005). Therefore, the presence of autoAb-autoAg complexes is likely to arise in certain patient populations (see Anderson and LaBaer, 2005).

In certain embodiments, provided herein are methods for pre-diagnosis detection of a pancreas hyperproliferative disorder by contacting a plurality or an array of antigen binding molecules with a test sample from a human subject having or suspected of having pancreas hyperproliferative disorder. The presence or absence of one or more autoantibody-autoantigen complexes in the test sample may indicate the presence or risk of disease, particularly when the level of the one or more (e.g., two to 20) autoantibody-autoantigen complexes in the test sample differs from the control sample.

In certain embodiments, any of the methods provided herein further comprise contacting a plurality or an array of antigen binding molecules with a test sample from a human subject at risk of developing a pancreas hyperproliferative disorder, wherein the test sample comprises autoantibody-autoantigen (autoAb-autoAg) complexes. The presence of one or more autoAb-autoAg complexes may indicate the risk of a pancreas hyperproliferative disorder when the level of the one or more autoAb-autoAg complexes in the test sample differs from a control, wherein at least one autoAb-autoAg complex in the test sample having a level that differs from the control comprises an autoantigen selected from BCL2L2, CCSP-2, COL24A1, CPB2, CRIP2, E2F4, GALNT1, GAS7, GRN, HNRPA2B1, IL15, INSR, ITGA1, LTBP1, PTGES3, RAD51, RAD52, RGS18, ST13, TGOLN2, or any combination thereof. In some embodiments, the methods disclosed herein include detecting autoAb-autoAg complexes, ERBB2 antigen, ESR1 antigen, TNC antigen, or any combination thereof. Accordingly, the methods disclosed herein include detecting autoAb-autoAg complexes, ERBB2 antigen, ESR1 antigen, and TNC antigen. In some embodiments, the methods disclosed herein include detecting autoAb-autoAg complexes, selected from BCL2L2, CCSP-2, COL24A1, CPB2, CRIP2, E2F4, GALNT1, GAS7, GRN, HNRPA2B1, IL15, INSR, ITGA1, LTBP1, PTGES3, RAD51, RAD52, RGS18, ST13, TGOLN2, the ERBB2 antigen, ESR1 antigen, and TNC antigen, or any combination thereof. Accordingly, the methods disclosed herein include detecting autoAb-autoAg complexes of BCL2L2, CCSP-2, COL24A1, CPB2, CRIP2, E2F4, GALNT1, GAS7, GRN, HNRPA2B1, IL15, INSR, ITGA1, LTBP1, PTGES3, RAD51, RAD52, RGS18, ST13, and TGOLN2, while at the same time (concurrently or sequentially) detecting the ERBB2 antigen, ESR1 antigen, and TNC antigen. In further embodiments, the methods disclosed herein include detecting autoAb-autoAg complexes of BCL2L2, CCSP-2, COL24A1, CPB2, CRIP2, E2F4, GALNT1, GAS7, GRN, HNRPA2B1, IL15, INSR, ITGA1, LTBP1, PTGES3, RAD51, RAD52, RGS18, ST13, TGOLN2, or any combination thereof, and also (concurrently or sequentially) detecting the ERBB2 antigen, ESR1 antigen, and TNC antigen.

In some embodiments, any of the methods disclosed herein further comprise detecting a glycosylation found on the antigens. Glycosylation includes structural changes of cell surface N- and O-glycans, such as sialylation, fucosylation, and the degree of branching. Representative glycosylations include a sialyl Lewis A (SLeA) or a sialyl Lewis X (SLeX). Methods for detecting SLeA and SLeX antigens are known in the art (see Rho et al. (2013) J. of Proteomics 96:291-99). As an example, antibodies directed to ERBB2, ESR1, and TNC are allowed to bind the respective biomarkers. Further, labeled anti-SLeA or anti-SLeX antibodies are incubated with the biomarkers. Biomarkers that are bound by both antibodies are then differentiated from antigens that are bound by only one antibody or no antibody.

In certain embodiments, the biomarkers are detected with a labeled anti-human immunoglobulin. In some embodiments, the anti-human immunoglobulin comprises a fluorescent label, such as a cyanine dye, a coumarin, a rhodamine, a xanthene, a fluorescein or sulfonated derivatives thereof, or a fluorescent protein. Alternately, the immunoglobulin can comprise a chromogenic reporter, such as horseradish peroxidase and an alkaline phosphatase. In some embodiments, the labeled anti-human immunoglobulin is an anti-IgA, anti-IgD, anti-IgE, anti-IgG, or anti-IgM.

In some embodiments, the biomarkers are detected with an antigen binding domain that is labeled with a tag molecule for use in vivo or in situ imaging. Accordingly, some embodiments include a tag molecule that has high contrast in MRI, ultrasound, X-ray, or PET imaging. An example of a high contrast MRI tag is the Gd-DTPA tag. Methods for tagging antibodies and imaging cancer with the Gd-DTPA tag are described in Zhang et al., Eur J Radiol 70:180-9; 2009 and Jun et al., Korean J Radiol 11:449-456, 2010, herein incorporated by reference in its entirety.

In other embodiments, the tag molecule is a microbubble or liposome, which can be used as a contrast agent in ultrasound. The microbubble or liposome has an antigen binding molecule incorporated into its shell, thereby allowing ultrasound imaging of tumors or cancer cells expressing the antigen. Methods for using microbubbles are described in Dayton et al., Mol Imaging; 3:125-34, 2004 and Lindner, Nat Rev Drug Discov 3:527-32, 2004, herein incorporated by reference in its entirety.

In some embodiments, the antigen binding molecule is labeled with a [¹⁸F]fluoro-2-D-deoxyglucose (FDG) tag molecule. Methods for using an FDG tagged antibody for PET imaging are described in Olafsen and Wu, Semin Nucl Med 40:167-81, 2010, herein incorporated by reference in its entirety.

In some embodiments, the antigen binding molecules are conjugated with a molecule that can be used to image cells or function as a therapeutic agent. Examples of imaging/therapeutic agent conjugates include Yttrium 90, Indium-111, or the like, as described in Lin and Iagaru, Curr Drug Discov Technol 7:253-62, 2010, herein incorporated by reference in its entirety. The binding domains can be used to image pancreatic cancers. In some embodiments, Yttrium 90 or Indium-111 labeled binding domains can be used as a targeted therapy for a pancreas hyperproliferative disorder.

Furthermore, any of the aforementioned methods can be combined with other known diagnostic methods for the disease of interest to further increase the sensitivity of the detection, diagnosis, prognosis or development of treatment regimens. For example, the methods can be performed in combination with an endoscopic ultrasound, endoscopic retrograde cholangiopancreatography (ERCP), computed tomography, magnetic resonance imaging, biopsy, or any combination thereof may be with the methods of the instant disclosure.

If the result of performing the methods described herein indicates an increased risk or diagnosis of a pancreas hyperproliferative disorder, a physician can then perform a biopsy on the human subject to confirm the presence of a pancreas hyperproliferative disorder.

In other embodiments, described herein are methods for treating a pancreas hyperproliferative disorder, comprising administering to a human subject an effective therapeutic regimen for a human subject, wherein the pancreas hyperproliferative disorder is detected in the subject by a method comprising identifying when a test sample from the human subject has at least one biomarker antigen that is elevated compared to a control. The level of biomarker antigen in the sample is measured by detecting the amount of biomarker antigen in the sample that specifically binds to an antigen binding domain. The biomarker antigen comprises at least one of an ERBB2 antigen, an ESR1 antigen, a TNC antigen, or any combination thereof. In certain embodiments, the level of at least two biomarker antigens is measured, such as ERBB2/ESR1, ERBB2/TNC, ESR1/TNC, or ERBB2/ESR1/TNC. In further embodiments, the level of a further biomarker antigen is measured, such as CA19-9 antigen, SEPT5, IL2RA, KRT16, GATA3, TLX3, CDK2AP1, STAT3, CLU, SERPINH1, HOXD13, BCL2, IL1A, MLLT10, DDB2, CD20, BRAF, STEAP2, PKM2, NDRG1, or any combination thereof. In some embodiments, the pancreas hyperproliferative disorder is a pancreatic ductal adenocarcinoma (PDA). In other embodiments, the pancreas hyperproliferative disorder is a precursor lesion.

Non-limiting examples of a therapeutic regimen include radiation therapy, chemotherapy, adjunctive therapy, surgery, or any combination thereof. For pancreatic cancer or a pancreatic cancer precursor lesion, several therapeutic regimens are known in the art. For example, the Whipple procedure, or pancreaticoduodenectomy, is the most commonly performed surgery to remove pancreatic tumors. Pancreatic cancer is considered resectable if the tumor appears to be localized to the pancreas without invasion into important surrounding structures, such as the mesenteric blood vessels (that supply blood to the intestines) located adjacent to the head portion of the pancreas. Furthermore there should be no evidence of metastatic spread to the liver or the intestinal lining. In a standard Whipple operation, a surgeon will remove the head of the pancreas, the gallbladder, part of the duodenum (i.e., the uppermost portion of the small intestine), a small portion of the stomach called the pylorus, and the lymph nodes near the head of the pancreas. Then the remaining pancreas and digestive organs are reconnected so that pancreatic digestive enzymes, bile, and stomach contents will flow into the small intestine during digestion. In another type of Whipple procedure, known as pylorus preserving Whipple, the bottom portion of the stomach, or pylorus, is not removed. In either case, such a surgery can last from about 6 hours to about 10 hours.

When pancreatic cancer has grown beyond the confines of the pancreas to invade surrounding vital structures, such a locally advanced pancreatic cancer is not treated by surgery. Treatment of locally advanced pancreatic cancer includes chemotherapy and radiation therapy. Exemplary chemotherapeutic drugs used for the treatment of pancreatic cancer include 5-fluorouracil, leukovirin, gemcitabine, cisplatin, irinotecan, paclitaxel, docetaxel, capecitabine, oxaliplatin and the FOLFIRINOX combination (5-fluorouracil, leucovorin, irinotecan and oxaliplatin). Exemplary radiation therapy is delivered in daily fractions over a six week period to a total dose of approximately 5,000 rads, which may be external (e.g, high energy X-rays) or internal (e.g., radiation contained in needles, seeds, wires, or catheters, which are placed directly into or near a tumor). In certain embodiments, chemotherapy may be administered together or sequentially with the radiation therapy.

Exemplary chemotherapeutic agents include alkylating agents (e.g., cisplatin, oxaliplatin, carboplatin, busulfan, nitrosoureas, nitrogen mustards, uramustine, temozolomide), antimetabolites (e.g., aminopterin, methotrexate, mercaptopurine, fluorouracil, cytarabine, gemcitabine), taxanes (e.g., paclitaxel, nab-paclitaxel, docetaxel), anthracyclines (e.g., doxorubicin, daunorubicin, epirubicin, idaruicin, mitoxantrone, valrubicin), bleomycin, mytomycin, actinomycin, hydroxyurea, topoisomerase inhibitors (e.g., camptothecin, topotecan, irinotecan, etoposide, teniposide), monoclonal antibodies (e.g., alemtuzumab, bevacizumab, cetuximab, gemtuzumab, panitumumab, rituximab, tositumomab, trastuzumab), vinca alkaloids (e.g., vincristine, vinblastine, vindesine, vinorelbine), cyclophosphamide, prednisone, leucovorin, oxaliplatin, hyalurodinases.

Within additional aspects of this disclosure, combination formulations and methods are provided comprising an effective therapeutic regimen for treating a disease of interest in combination with one or more secondary or adjunctive therapies. Such therapies may be additional active agents that are formulated together or administered coordinately with the known treatments of the disease of interest. Useful adjunctive or neoadjunctive therapies for combinatorial formulation or coordinate treatment methods include, for example, enzymatic nucleic acid molecules, allosteric nucleic acid molecules, antisense, decoy, or aptamer nucleic acid molecules, antibodies such as monoclonal antibodies, small molecules and other organic or inorganic compounds including metals, salts and ions, steroids, non-steroidal anti-inflammatory drugs (NSAIDs), and other drugs or active agents or procedures indicated for treating a particular disease, including surgery, chemotherapy, radiation therapy, chemoradiation therapy, or the like.

To practice the coordinate administration methods, a particular treatment may be administered simultaneously or sequentially in a coordinated treatment protocol with one or more secondary or adjunctive therapies. The coordinate administration may be done in either order, and there may be a time period while only one or both (or all) therapies, individually or collectively, exert their biological activities. A distinguishing aspect of all such coordinate treatment methods is that a composition elicits some favorable clinical response, which may or may not be in conjunction with a secondary clinical response provided by the secondary therapeutic agent.

In certain embodiments, any of the methods described herein include a human subject that is at risk for developing a pancreas hyperproliferative disorder. In some embodiments, a subject is at risk because the subject belongs to a subpopulation identified by specific characteristics, such as age, gender, diet, ethnicity, family history, or a combination thereof. Members of at risk populations include, for example, cigarette smokers or individuals with at least 1, 2, 3, or more first degree relatives that have been diagnosed with pancreatic cancer. In some embodiments, the subject is at risk if the subject has a mutation in a gene or heritable disease such as, for example, BRCA1, BRCA2, P16/INK4A, TP53 (e.g., Li-Fraumeni syndrome), palladin (PALLD), familial atypical multiple mole melanoma syndrome (FAMMM), hereditary pancreatitis (PRSS1), Peutz-Jeghers Syndrome (LKB1/STK11) or hereditary non-polyposis colorectal cancer syndrome (HNPCC).

In some embodiments, the methods described herein can be used in the design of genetically engineered T-cell therapies that target ERBB2, ESR1, TNC or a combination thereof. The markers identified herein can be used as targets to develop high-affinity T-cell receptors (TCR) that can be utilized in immunotherapies (e.g., adoptive immunotherapy or TCR gene therapy). TCR gene therapy is a treatment approach designed to overcome obstacles associated with conventional T cell adoptive immunotherapy, such as the extensive time and labor required to isolate, characterize, and expand tumor antigen-specific T cells (Schmitt et al., Hum. Gene Ther. 20:1240, 2009). Strategies are known to enhance the affinity of TCRs intended for use in TCR gene therapy (Udyavar et al., J. Immunol. 182:4439, 2009; Zhao et al., J. Immunol. 179:5845, 2007; Richman and Kranz, Biomol. Eng. 24:361, 2007). These approaches generally entail generating libraries of mutated TCR genes and subsequent screening for mutations that confer higher affinity for the complex of target peptide with major histocompatibility complex (MHC) ligand. Mutations are usually targeted to the complementarity determining regions (CDRs) known to interact with the peptide (CDR3) and/or MHC (CDR1/2) (Wucherpfennig et al., Cold Spring Harb. Perspect. Biol. 2:a005140, 2010). In this way, T-cells are generated that specifically target cells that over-express target antigens such as ERBB2, ESR1, or TNC and thereby target pancreas hyperproliferative disorder.

In certain embodiments, the methods described herein can be used to design chimeric antigen receptors (CAR). A CAR is an engineered TCR that has had the specificity of a binding molecule (e.g., a monoclonal antibody) grafted onto the TCR. The sequence encoding the specificity can be introduced into a T-cell via a retroviral vector via methods known in the art (Lipowska-Bhalla et al., Cancer Immunol Immunother. 61:953-62, 2012). Accordingly, CARs specific to the biomarker antigens disclosed herein (e.g., ERBB2, ESR1, TNC) can be generated and used as a therapeutic for the treatment of a pancreas hyperproliferative disorder.

In certain embodiments, any of the methods described herein have a specificity that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. In certain embodiments, any of the methods described herein have a sensitivity of at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, any of the methods described herein have a specificity for pancreatic cancer that is about 80% and a sensitivity that ranges from about 50% to about 95%. In some embodiments, any of the methods described herein have a specificity for pancreatic cancer that is about 90% and a sensitivity that ranges from about 50% to about 95%. In certain embodiments, any of the methods described herein have a specificity for pancreas cancer that is about 90% and a sensitivity of at least 30% in, for example, a subject having a high risk of having a pancreas hyperproliferative disease. In some embodiments, any of the methods described herein have a specificity for pancreatic cancer that is about 95%, 96%, 97%, 98%, or 99% and a sensitivity that is about 80%, 81%, 82%, 83%, 84%, 85%, 86%, 87%, 88%, 89%, 90%, 91%, 92%, 93%, 94%, 95%, 96%, 97%, 98%, or 99%. In certain embodiments, any of the methods described herein have a specificity for pancreas cancer that is about at least 99% and a sensitivity of at least about 90% in, for example, a subject that has not been associated with a risk factor for a pancreas hyperproliferative disease.

As used herein, “sensitivity” refers to the proportion of subjects (e.g., humans) that have a disease and test positive over the total population that have the disease (usually expressed as a percentage). For example, a human patient population that has pancreatic cancer and detection of ERBB2, ESR1, or TNC will be a measure of the proportion of actual pancreatic cancer positives that are correctly identified as such (e.g., the percentage of pancreatic cancer patients who are correctly identified as having the condition). In other words, “high sensitivity” means there are few false negatives present and “low sensitivity” means there are many false negatives present.

As used herein, “specificity” refers to a measure of the proportion of subjects (e.g., humans) that correctly test negative for the disease over the total population of subjects that do not have the disease. For example, a human patient population that has pancreatic cancer and detection of ERBB2, ESR1, or TNC measures the proportion of negatives which are correctly identified as such (e.g., the percentage of healthy people who are correctly identified as not having the condition). In other words, “high specificity” means there are few false positives present and “low specificity” means there are many false positives present.

In another aspect, the present invention provides kits comprising materials useful for carrying out diagnostic methods according to the present invention. The diagnosis procedures described herein may be performed by diagnostic laboratories, experimental laboratories, or practitioners. The invention provides kits, which can be used in these different settings. Materials and reagents for characterizing biological samples and diagnosing a pancreas hyperproliferative disease in a subject according to the methods herein may be assembled together in a kit. In certain aspects, a kit comprises at least one reagent that specifically detects levels of one or more biomarkers disclosed herein, and instructions for using the kit according to a method of this disclosure.

Each kit may preferably include the reagent (e.g., primary antibody specific for a biomarker, labeled anti-human immunoglobulin) that renders the procedure specific. Thus, for detecting/quantifying a biomarker, the reagent that specifically detects levels of the biomarker may be an antibody that specifically binds to the antigen of interest. A kit of the present disclosure may further comprise one or more substrates to anchor the antigen binding molecules, including microarray slides, beads, plastic tubes, or other surfaces, one or more antibodies to biomarker, labeling buffer or reagents, wash buffers or reagents, immunodetection buffer or reagents, and detection means. Protocols for using these buffers and reagents for performing different steps of the procedure may be included in the kit. The reagents may be supplied in a solid (e.g., lyophilized) or liquid form. The kits of the present disclosure may optionally comprise different containers (e.g., slide, vial, ampoule, test tube, flask or bottle) for each individual buffer or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain embodiments, kits of the present disclosure further include control samples, control slides, or both. Instructions for using the kit, according to one or more methods of this disclosure, may comprise instructions for processing the biological sample obtained from a subject, or for performing the test, instructions for interpreting the results. As well as a notice in the form prescribed by a governmental agency (e.g., FDA) regulating the manufacture, use or sale of pharmaceuticals or biological products.

### EXAMPLES

### Example 1

### Sample Collection and Analysis

By way of background, here we used our high density antibody microarray platform customized for interrogation of pancreas cancer samples, and applied it to: 1) plasma drawn from the highly faithful KPC mouse model of pancreas cancer; 2) pre-diagnostic plasma from women who later succumbed to PDA; and 3) to diagnostic plasma from patients, to discover and preliminarily validate putative early disease markers of PDA. Focusing on plasma membrane and secreted proteins identified as up-regulated by our array experiments, our approach identified two markers overlapping between mouse and pre-diagnostic human datasets that have been previously implicated in PDA; and a third marker, ESR1, was identified by multiple distinct antibodies as elevated in the pre-diagnostic human plasma samples. In a subsequent set of array experiments on 24 diagnostic PDA compared to 24 control plasma samples, all 3 of these markers were again up-regulated, collectively providing preliminary confirmation across multiple sample sets. The implications of our findings and their applicability to early and clinically meaningful diagnosis of pancreas cancer are further discussed for this 3-protein panel of biomarkers.

### Pre-Diagnostic Patient Samples

Eighty-seven pre-diagnostic PDA and 87 matched control plasma samples collected in EDTA were obtained from the Women's Health Initiative's (WHI) observational study. Controls were matched 1:1 to PDA cases based on the following criteria: age at screening, year of WHI enrollment, alcohol consumption at baseline, race/ethnicity, smoking status (never, past, current), diabetes history (yes or no), prior hormone replacement therapy (none, estrogen only, estrogen and progesterone), blood draw visit (baseline only, baseline and year 3, year 3 only) and follow-up duration (i.e., controls must have been followed and lived for at least the same interval between enrollment and pancreas cancer diagnosis as their matched cases). A reference pool of EDTA-collected plasma was created by pooling plasma drawn from a group of seven female volunteers from the Fred Hutchinson Cancer Research Center, aged 27-45. All samples were de-identified and the study was approved by the FHCRC Institutional Review Board.

### Diagnostic Patient Samples

Twenty-four diagnostic EDTA-collected plasma samples were provided by the Center for Accelerated Translation in Pancreas Cancer (CATPAC) at the Seattle Cancer Care Alliance, Seattle, Wash. Unmatched control (not diagnosed with any cancer) plasma samples were collected and processed at the same clinic using the same methods. All patient information provided was done so in accordance with the Institutional Review Board at the Fred Hutchinson Cancer Research Center.

### Mouse Plasma Collection

Murine plasma samples were acquired from Kras^{LSL-G12D/+}; Trp53^{R172H/+}; Pdx-1Cre or p48^{Cre/+} (KPC) and age-matched KP and Cre control mice on a mixed C57BL/6/SV129 mixed background following brief anesthesia with isoflurane. Both Pdx-1 and p48 promoters were used to drive Cre expression and target pancreas-specific expression of the point mutant alleles. Blood (maximum 200 μl) was collected every two weeks from KPC and age-matched control mice into tubes containing 10 μl 0.5M EDTA. Samples were centrifuged (4015 g×10 minutes), the supernatant collected and re-centrifuged (16,060 g) for an additional 10 minutes, and aliquots were then stored at −80° C. until use in the array experiments.

### Mouse Tissue Collection and Induction of Chronic Pancreatitis

Tissue from the head of the pancreas of 2-month old, 4-month old and end-stage disease KPC and age-matched KP and Cre control animals was collected at necropsy and flash frozen until use in antibody microarray experiments. Tissues were similarly harvested from a cohort of six 2-month old WT mice injected intraperitoneally with 100 μl cerulein (50 μg/ml) for 23 consecutive days to induce chronic pancreatitis and necropsies were performed within 6 hours of the final injection. All mouse husbandry and procedures were conducted in accordance with a protocol approved by the Institutional Animal Care and Use Committee at the Fred Hutchinson Cancer Research Center.

### Sample Preparation for Antibody Microarray Analysis

Plasma samples were depleted of IgG and serum albumin using the Proteoprep Immunoaffinity Albumin and IgG Depletion Kit per the manufacturer's instructions (Sigma Aldrich, St. Louis, Mo.). Depleted case and control plasma and murine tissue lysates (250 μg of total protein for pre-diagnostic, diagnostic and murine plasma samples and 200 μg for murine tissue samples) were labeled with N-hyroxysuccinimide (NHS)-Cy5 (GE Health Biosciences, Pittsburgh, Pa.) and a pool of reference plasma and tissue lysates collected from wild type mice with NHS-Cy3.

KPC and age-matched matched control pancreatic tissue collected at 2 months, 4 months and end-stage disease were weighed and lysed in a 10:1 ratio of lysis buffer to tissue weight using 1% NP-40, 0.25% deoxycholate, 0.25% octyl-β-d-glucopyranoside and 0.25% amidosulfobetaine-14 supplemented with phosphatase inhibitors, Roche protease inhibitor cocktail (Roche USA, Indianapolis, Ind.) and 1 mM PMSF.

### Antibody Microarray Experiments (Printing and Hybridization)

Antibody microarray slides were printed and labeled plasma and tissue samples incubated on Nexterion slide H (Schott, Germany) arrays similarly to our previously described methods (Ramirez et al., Mol Cell Proteomics 9:1449-60, 2010). Briefly, antibodies were printed in triplicate in a 16×16 block format with 48 blocks per array for a total of 3×4096 unique features. Antibodies were printed at a final concentration of 175-350 μg/ml unless their initial concentrations were lower. Following a “case/control versus reference” procedure, individual Cy5-labeled case and control samples were pooled with an equal amount of Cy3-labeled reference to remove dye bias from the analyses. Labeled lysates were incubated on arrays for 1.5 hours, and following a series of washes to remove excess dye, arrays were scanned and analyzed using an Axon Genepix 4200A scanner (Molecular Devices, LLC, Sunnyvale, Calif.).

### Array Analyses and Statistics

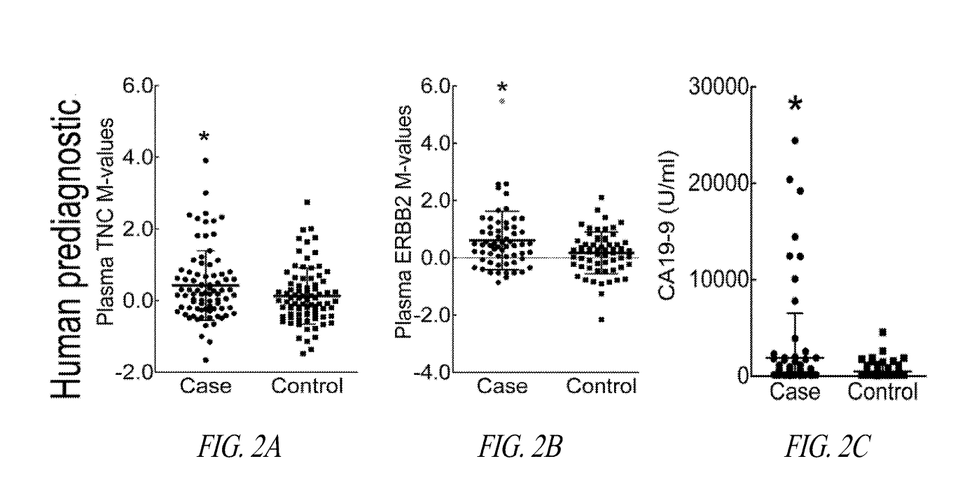

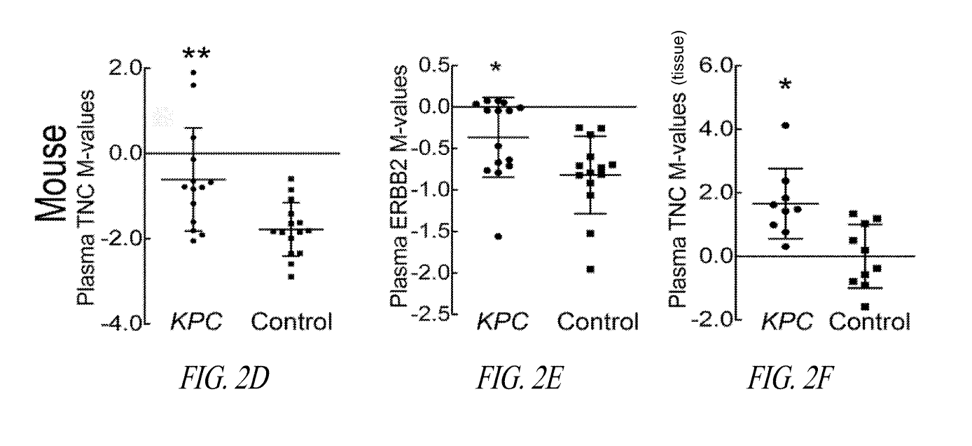

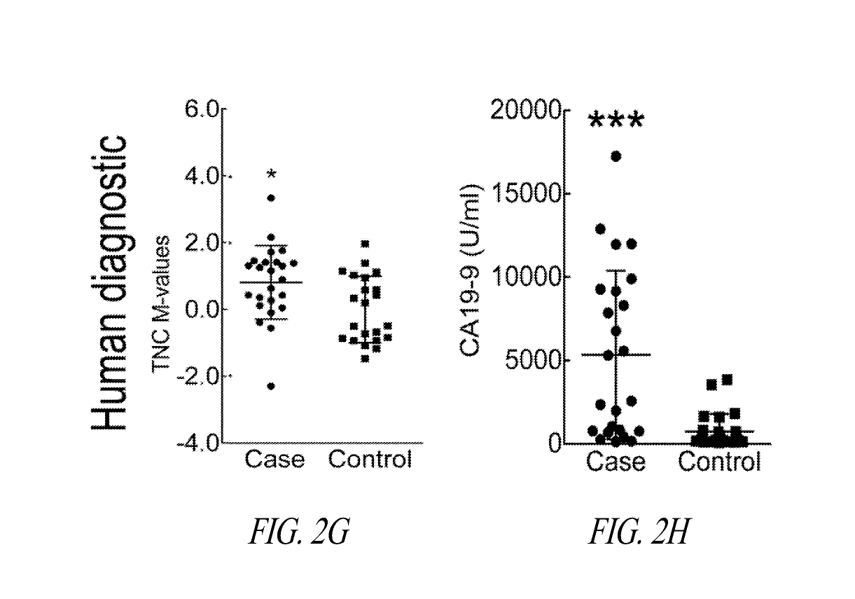

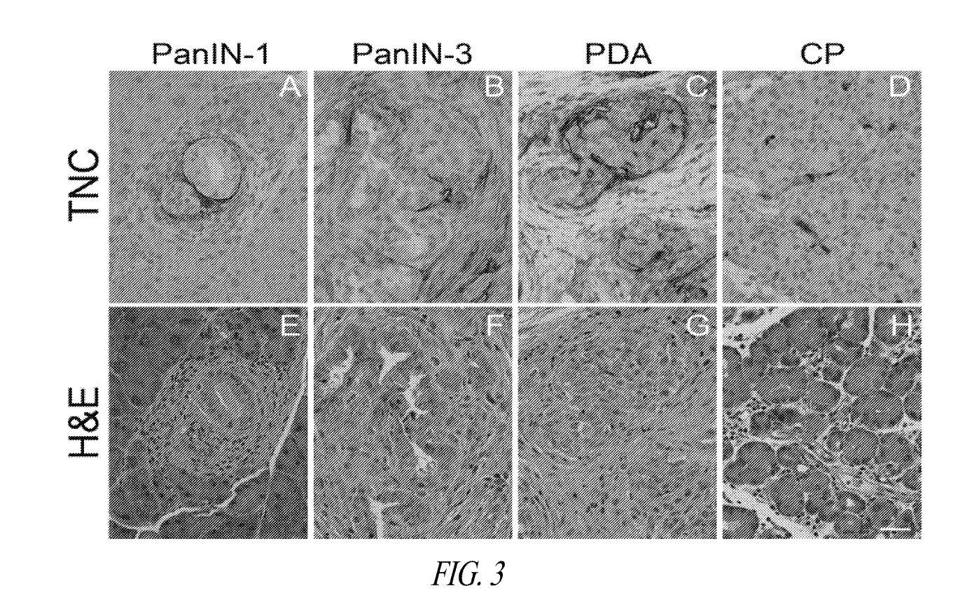

For each antibody, difference in case/control signal (red channel) compared to reference (green channel), known as the M value, was calculated as log₂(R_{c}/G_{c}); where R_{c} is red corrected, and G_{c} is green corrected (using the normexp background correction method developed by Smyth et al., Methods 31:265-73, 2003). Saturated array spots were flagged and triplicate antibodies with coefficients of variation >10% were removed prior to array normalization. Following localized background correction, print tip loess intra-array normalization was performed. Inter array green channel quantile adjustment was then applied to normalize the reference (green) signal. An average of the median intensity of triplicate spots for each antibody feature was calculated and control signal was subsequently standardized to have a mean signal of zero with a standard deviation of 1. For the WHI pre-diagnostic plasma dataset using the 87 cases and 87 matched controls, linear regression, which considers covariate effects such as hybridization day, print day and body mass index (BMI), was used. Paired t-tests were conducted for the adjusted measures, which is calculated as ‘estimated.m=m−BETA*(hybridization day+print day+BMI)’, where the hybridization day, print day and BMI are all factor categories. For the diagnostic plasma dataset, linear regression was fitted considering only the hybridization days; for the mouse plasma and tissue data, we used logistic regression ‘logit(case/control)˜est.m’, where the est.m is the estimated assay after removing the hybridization day effect. Candidate protein markers were then ranked based on their p-values and log₂ odds ratios (WHI and CATPAC) or logistic regression odds ratio (mouse plasma and tissue). A positive odds ratio (OR) means the candidate protein is greater in cancer than controls; a negative value means the converse. All normalization procedures and analyses were conducted using R statistical computing software program. CA19-9 was analyzed at the University of Pittsburgh Luminex Core Facility (www.anes.upmc.edu/research/facilities/luminex_core_facility.aspx). The statistical analyses presented in FIG. 2 used a paired 2-tailed t-test for the WHI samples and unpaired 2-tailed t-tests for the murine and CATPAC diagnostic sample sets, respectively, and were computed using GraphPad Prism 5.0.

### Immunohistochemistry

Four-micron sections of formalin-fixed paraffin-embedded tissues were cut and incubated for 1 hr at 60′C. Sections were deparaffinized with xylene and rehydrated sequentially. Antigen retrieval was performed in Trilogy pH 8.0 Buffer (Cell Marque, Rocklin, Calif.) in a pressure cooker and subsequent incubations performed on a Dako Autostainer Plus (Agilent Technologies, Santa Clara, Calif.). Slides were treated with 3% hydrogen peroxide and blocked with TCT Buffer (0.05M Tris, 0.15M NaCl, 0.25% Casein, 0.1% Tween 20, pH 7.6). Tissues were then incubated with anti-TNC antibody (1:75) (Novus Biologicals, Littleton, Colo.) or a matched concentration of rabbit IgG. Poly-HRP anti-Rabbit IgG Polymer (Leica Microsystems, Buffalo Grove, Ill.) was then applied followed by DAB+substrate-chromagen (Agilent. Technologies, Santa Clara, Calif.). Slides were counterstained with hematoxylin (Agilent). Serial sections were stained with hematoxylin and eosin for histological analysis. All images were captured with a Nikon DS-Vi1 brightfield camera using NIS Elements 3.2 Basic Research Image software (Nikon Instruments Inc., Melville, N.Y.).

### Example 2

### Antibody Microarray Interrogation of Murine Pre-Invasive and Invasive Plasma

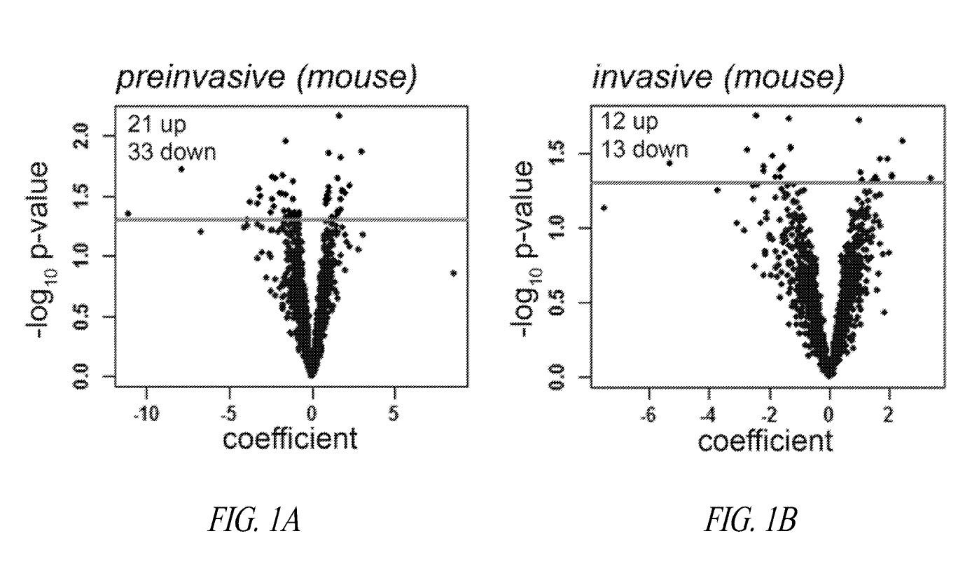

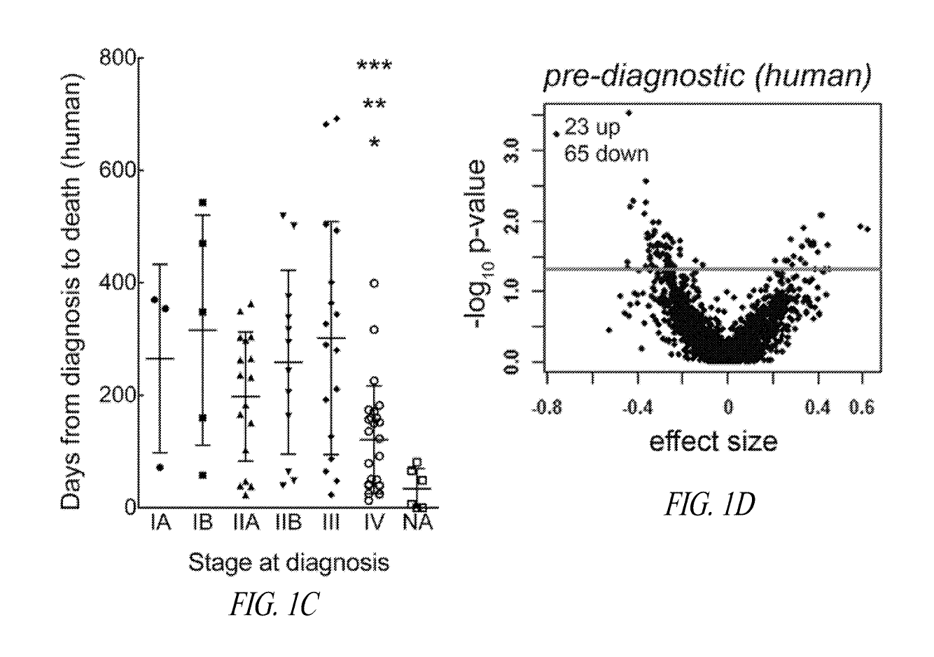

To discover putative diagnostic biomarkers associated with early stages of pancreas cancer (i.e., precursor lesions or resectable cancers), we elected to use plasma collected from the highly faithful KPC mouse model of PDA. These mice recapitulate the clinical, histological and molecular characteristics of the human disease with 100% penetrance and have been used extensively in preclinical therapeutic studies (Hingorani et al., Cancer Cell 7:469-83, 2005; Olive et al., Science 324:1457-61, 2009; Provenzano et al. Cancer Cell 21:418-29, 2012; Beatty et al., Science 331:1612-6, 2011). Plasma samples collected at 6-8 weeks representing pre-invasive disease stages (time point #1, or TP1), and mid-way through the lifespan of each individual KPC mouse (time point #2, or TP2) representing early invasive adenocarcinoma, were compared to controls on an antibody array platform containing 4,096 unique features. This platform was tailored for interrogation of pancreas cancer tissue and plasma samples by including approximately 130 antibodies based on their relevance to PDA. These included antibodies against proteins identified as markers of PDA (see Harsha et al., PLoS Med 6:e1000046, 2009), with further enrichment for putative early detection markers of PDA, i.e., markers identified in higher-grade precursor pancreatic intraepithelial neoplasia 3 (PanIN-3) lesions. Unpaired logistic regression analyses identified fifty-four proteins at TP1 (21 proteins that were up regulated in KPC plasma versus controls and 33 down regulated) and 25 proteins at TP2 (12 up, 13 down) (FIG. 1A, 1B and Table 1) that differentiated KPC from control plasma with statistical significance (p-value <0.05, none are significant if multiple comparison testing is performed given the high dimensionality of the array). Among the up-regulated markers were the plasma membrane proteins IL12RB2, AQP2, PCDH15, ICAM5, OPN3, CD27 (from TP1) and RAB7L1, EZR, ERBB2, CCR2 and CSF3R (from TP2); and the extracellular or secreted markers TNC, HBA1, PTHLH (from TP1) and B2M and SERPING1 (from TP2).

TABLE 1
Candidate plasma biomarkers from analysis of KPC TP1 and TP2 (pre-
invasive and invasive) plasma antibody microarray interrogation
Time point 1 (TP1)

| | | Coef- | p- |
|---|---|---|---|
| | | ficient | value |
| Gene name | Protein name | TP1 | TP1 |
| TP53 | tumor protein p53 | 1.66 | 0.0068 |
| ANXA2 | annexin A2 | −1.55 | 0.0113 |
| IL12RB2 | interleukin 12 receptor, beta 2 | 2.96 | 0.0135 |
| AQP2 | aquaporin 2 (collecting duct) | 1.05 | 0.0140 |
| TNC | tenascin C | 1.76 | 0.0153 |
| CREB1 | cAMP responsive element binding | −7.85 | 0.0190 |
| | protein 1 | | |
| VIL2 (EZR) | Ezrin | −1.75 | 0.0212 |
| NFKB1 | Nuclear Factor Of Kappa Light | −2.37 | 0.0222 |
| | Polypeptide Gene Enhancer In B- | | |
| | Cells 1 | | |
| FOS | FBJ murine osteosarcoma viral | 1.57 | 0.0225 |
| | oncogene homolog | | |
| SPINK1 | serine peptidase inhibitor, Kazal type 1 | −2.17 | 0.0227 |
| PTEN | phosphatase and tensin homolog | −1.15 | 0.0237 |
| AKAP12 | A kinase (PRKA) anchor protein 12 | 2.24 | 0.0264 |
| UBE2S | ubiquitin-conjugating enzyme E2S | 0.98 | 0.0272 |
| PLCG1 | phospholipase C, gamma 1 | −3.16 | 0.0278 |
| KI67 | monoclonal recognizing Ki-67 | 1.79 | 0.0281 |
| APAF1 | apoptotic peptidase activating factor 1 | 1.86 | 0.0291 |
| AMBRA1 | autophagy/beclin-1 regulator 1 | 1.03 | 0.0296 |
| MKI67 | monoclonal recognizing Ki-67 | 2.00 | 0.0301 |
| ADAM28 | ADAM metallopeptidase domain 28 | −1.82 | 0.0302 |
| YWHAZ | tyrosine 3- | −1.94 | 0.0303 |
| | monooxygenase/tryptophan 5- | | |
| | monooxygenase activation protein, | | |
| | zeta polypeptide | | |
| FANCF | Fanconi anemia, complementation | 0.96 | 0.0309 |
| | group F | | |
| WDR1 | WD repeat domain 1 | −1.60 | 0.0313 |
| IRS1 | insulin receptor substrate 1 | −3.10 | 0.0317 |
| MAPK3/ | mitogen-activated protein kinase | −1.16 | 0.0333 |
| MAPK1 | 1/mitogen-activated protein kinase 3 | | |
| (pThr202/ | | | |
| Tyr204) | | | |
| FTH1 | ferritin, heavy polypeptide 1 | 1.77 | 0.0333 |
| PCDH15 | protocadherin-related 15 | 0.98 | 0.0335 |
| HBA1 | hemoglobin, alpha 1 (CD31) | 0.83 | 0.0336 |
| STAT3 | signal transducer and activator of | −2.43 | 0.0341 |
| | transcription 3 (acute-phase response | | |
| | factor) | | |
| RPS5 | ribosomal protein S5 | −3.73 | 0.0360 |
| PIK3CA | phosphoinositide-3-kinase, catalytic, | 0.88 | 0.0362 |
| | alpha polypeptide | | |
| LASP1 | LIM and SH3 protein 1 | −3.26 | 0.0368 |
| FKBP4 | FK506 binding protein 4, 59 kDa | −2.27 | 0.0393 |
| ICAM5 | intercellular adhesion molecule 5, | 1.70 | 0.0406 |
| | telencephalin | | |
| PRKAR2A | protein kinase, cAMP-dependent, | −1.36 | 0.0419 |
| | regulatory, type II, alpha | | |
| TIMP1 | TIMP metallopeptidase inhibitor 1 | −1.70 | 0.0421 |
| GADD45G | growth arrest and DNA-damage- | 1.54 | 0.0429 |
| | inducible, gamma | | |
| DPYSL3 | dihydropyrimidinase-like 3 | −1.00 | 0.0433 |
| CLU | Clusterin | −0.77 | 0.0434 |
| THBS2 | thrombospondin 2 | −1.07 | 0.0434 |
| LASP1 | LIM and SH3 protein 1 | −2.46 | 0.0440 |
| OPN3 | opsin 3 | 1.57 | 0.0448 |
| ABL1 | c-abl oncogene 1, non-receptor | −11.12 | 0.0454 |
| | tyrosine kinase | | |
| IL1B | interleukin 1, beta | −1.32 | 0.0464 |
| IL5 | interleukin 5 (colony-stimulating | −1.22 | 0.0464 |
| | factor, eosinophil) | | |
| MT3 | metallothionein 3 | −0.79 | 0.0466 |
| EPRS | glutamyl-prolyl-tRNA synthetase | −1.61 | 0.0469 |
| PTHLH | parathyroid hormone-like hormone | 1.24 | 0.0470 |
| TPI1 | triosephosphate isomerase 1 | −1.24 | 0.0475 |
| KLK5 | kallikrein-related peptidase 5 | −0.88 | 0.0477 |
| REG4 | regenerating islet-derived family, | −1.45 | 0.0480 |
| | member 4 | | |
| CD27 | CD27 molecule | 1.40 | 0.0483 |
| NTNG1 | netrin G1 | −1.48 | 0.0493 |
| RAD54L | RAD54-like (S. cerevisiae) | −3.95 | 0.0497 |
| AFP | alpha-fetoprotein | −1.30 | 0.0500 |

Time point 2 (TP2)

| | | Coef- | p- |
|---|---|---|---|
| | | ficient | value |
| Gene name | Protein name | TP2 | TP2 |
| FANCF | Fanconi anemia, complementation | 1.01 | 0.0193 |
| | group F | | |
| B2M | beta-2-microglobulin | 2.44 | 0.0264 |
| RAB7L1 | RAB7, member RAS oncogene | 1.68 | 0.0343 |
| | family-like 1 | | |
| | Ezrin | 1.92 | 0.0344 |
| NF2 | neurofibromin 2 (merlin) | 1.03 | 0.0428 |
| ERBB2 | v-erb-b2 erythroblastic leukemia viral | 2.09 | 0.0443 |
| | oncogene homolog 2, | | |
| | neuro/glioblastoma derived oncogene | | |
| | homolog (avian) | | |
| CCR2 | chemokine (C-C motif) receptor 2 | 1.55 | 0.0451 |
| LIN13 | unknown gene (C. elegans) | 2.10 | 0.0459 |
| SUFU | suppressor of fused homolog | 3.38 | 0.0472 |
| | (Drosophila) | | |
| SERPING1 | serpin peptidase inhibitor, clade G | 1.58 | 0.0475 |
| | (C1 inhibitor), member 1 | | |
| CSF3R | colony stimulating factor 3 receptor | 1.09 | 0.0478 |
| | (granulocyte) | | |
| MCM6 | minichromosome maintenance | 1.44 | 0.0485 |
| | complex component 6 | | |
| RELA | v-rel reticuloendotheliosis viral | −2.42 | 0.0177 |
| | oncogene homolog A (avian) | | |
| ALPPL2 | alkaline phosphatase, placental-like 2 | −1.34 | 0.0188 |
| FANCF | Fanconi anemia, complementation | 1.01 | 0.0193 |
| | group F | | |
| B2M | beta-2-microglobulin | 2.44 | 0.0264 |
| BAD | BCL2-associated agonist of cell death | −1.29 | 0.0289 |
| SPINT2 | serine peptidase inhibitor, Kunitz | −1.30 | 0.0293 |
| | type, 2 | | |
| BCR | breakpoint cluster region | −2.70 | 0.0298 |
| FABP3 | fatty acid binding protein 3, muscle | −1.87 | 0.0331 |
| | and heart (mammary-derived growth | | |
| | inhibitor) | | |
| RAB7L1 | RAB7, member RAS oncogene | 1.68 | 0.0343 |
| | family-like 1 | | |
| EZR | Ezrin | 1.92 | 0.0344 |
| SMAD2 | SMAD family member 2 | −5.31 | 0.0372 |
| MET | met proto-oncogene (hepatocyte | −2.18 | 0.0387 |
| | growth factor receptor) | | |
| PTPRF | protein tyrosine phosphatase, receptor | −1.51 | 0.0389 |
| | type, F | | |
| POLE4 | polymerase (DNA-directed), epsilon | −1.63 | 0.0403 |
| | 4, accessory subunit | | |
| EIF4E | eukaryotic translation initiation factor | −2.17 | 0.0414 |
| | 4E | | |
| NF2 | neurofibromin 2 (merlin) | 1.03 | 0.0428 |
| ERBB2 | v-erb-b2 erythroblastic leukemia viral | 2.09 | 0.0443 |
| | oncogene homolog 2 | | |
| CCR2 | chemokine (C-C motif) receptor 2 | 1.55 | 0.0451 |
| CCNA2 | cyclin A2 | −1.62 | 0.0452 |
| HAPLN1 | hyaluronan and proteoglycan link | −1.79 | 0.0457 |
| | protein 1 | | |
| LIN13 | lin13 (C. elegans) | 2.10 | 0.0459 |
| SUFU | suppressor of fused homolog | 3.38 | 0.0472 |
| | (Drosophila) | | |
| SERPING1 | serpin peptidase inhibitor, clade G | 1.58 | 0.0475 |
| | (C1 inhibitor), member 1 | | |
| CSF3R | colony stimulating factor 3 receptor | 1.09 | 0.0478 |
| | (granulocyte) | | |
| MCM6 | minichromosome maintenance | 1.44 | 0.0485 |
| | complex component 6 | | |

Candidate biomarkers for KPC pre-invasive and invasive plasma drawn between 8-10 weeks and mid-way through disease progression, respectively. Antibody microarray interrogation and logistic regression analyses were used to identify candidate plasma proteins differentiating KPC from age-matched control plasma samples with statistical significance. Plotted are markers with p-value <0.05 and their normalized odds ratios, representing the degree to which each marker is elevated or down regulated in KPC versus control plasma. Candidates are listed based on ascending p-values.

Therefore, the array experiments performed on samples collected from KPC mice identified a number of plasma membrane and secreted proteins that are potential markers for use in the detection and diagnosis of pancreatic cancer in humans.

### Example 3

### Antibody Microarray Interrogation of Pre-Diagnostic Human Plasma Samples

To identify putative protein markers that could be used to detect PDA in asymptomatic individuals, we interrogated pre-diagnostic plasma samples drawn from a large cohort of subjects who succumbed to PDA within 4 years of the blood draw and matched controls (see Example 1 for matching criteria and Table 2 for sample characteristics). This cohort of 87 cases and matched control samples represent, to our knowledge, the largest set of pre-diagnostic pancreas cancer plasma samples interrogated to discover early detection biomarkers of PDA.

TABLE 2
Patient characteristics for WHI pre-diagnostic cases and
controls and the CATPAC diagnostic cases
WHI

| | Cases | Controls |
|---|---|---|
| Age | | |
| 50-59 | 14 | 14 |
| 60-69 | 32 | 33 |
| 70-80 | 41 | 40 |
| Ethnicity | | |
| Asian | 4 | 5 |
| Black | 4 | 3 |
| White | 76 | 76 |
| Other | 3 | 3 |
| Smoking status | | |
| Never | 40 | 70 |
| Current | 8 | 7 |
| Past | 39 | 40 |
| HRT status | | |
| Estrogen alone | 15 | 17 |
| Estrogen + | 16 | 16 |
| progesterone | | |
| None | 56 | 54 |
| BMI | | |
| Normal | 29 | 38 |
| Overweight | 35 | 27 |
| Obese | 22 | 21 |
| NA | 1 | 1 |

State at dignosis for WHI cases

| Stage | N |
|---|---|
| IA | 3 |
| IB | 5 |
| IIA | 19 |
| IIB | 12 |
| III | 17 |
| IV | 26 |
| Unknown | 5 |
| | 87 |

CATPAC

| Age at diagnosis | Cases |
|---|---|
| 30-49 | 4 |
| 50-59 | 7 |
| 60-69 | 7 |
| 70-80 | 6 |

Sex

| Male | Female |
|---|---|
| 17 | 7 |

| | Cases |
|---|---|
| Smoking status | |
| Never | 7 |
| Current | 4 |
| Past | 12 |
| N.A. | 1 |
| BMI | |
| Normal | 9 |
| Overweight | 9 |
| Obese | 6 |

State at diagnosis for CATPAC cases

| Stage | N |
|---|---|
| IA | 1 |
| IB | 2 |
| IIA | 4 |
| IIB | 6 |
| III | 7 |
| IV | 2 |
| Unknown | 2 |
| | 24 |

The time from blood draw to diagnosis for this sample set ranged from 33 days to just under 4 years, and the time from diagnosis to death ranged from 0 to just under 700 days. The stage determined at the time of diagnosis was not significantly correlated with time to death except for stage IV (i.e., metastatic) disease (based on using M=1 in the T, N, M staging system), which comprised 30% of our sample patient population (FIG. 1C). Thus, samples were derived from patients diagnosed at different stages of disease and reflected the expected heterogeneity of PDA based on the ranges in days to death following diagnosis. A paired t-test identified a total of 88 candidate markers differentiating pre-diagnostic plasma samples from controls with statistical significance (p-value <0.05, none are significant if multiple comparison testing is considered). Twenty-three of these markers were up-regulated and 65 were down-regulated (FIG. 1D). The complete list of candidate early detection markers is provided in Table 3. The median inter-array variation across 27 arrays incubated with replicate samples in a blinded manner was 0.043 (range 0.0009-0.29), showing strong reproducibility across individual arrays.

TABLE 3
Candidate plasma biomarkers from analysis of WHI pre-diagnostic plasma
antibody microarray interrogation

| Gene Name | Protein name | Effect size | p-value |
|---|---|---|---|
| DPP10 | dipeptidyl-peptidase 10 (non-functional) | −0.43 | 0.0003 |
| ALDH1A1 | aldehyde dehydrogenase 1 family, member A1 | −0.76 | 0.0006 |
| AAMP | angio-associated, migratory cell protein | −0.36 | 0.0028 |
| PCTK1 | cyclin-dependent kinase 16 | −0.41 | 0.0052 |
| (CDK16) | | | |
| BCL3 | B-cell CLL/lymphoma 3 | −0.36 | 0.0054 |
| EIF2C2 | argonaute RISC catalytic component 2 | −0.43 | 0.0064 |
| (AGO2) | | | |
| ITGB1 | integrin, beta 1 | −0.36 | 0.0079 |
| SEPT5 | septin 5 | 0.42 | 0.0083 |
| IL2RA | interleukin 2 receptor, alpha | 0.42 | 0.0085 |
| MAP2K3 | mitogen-activated protein kinase kinase 3 | −0.33 | 0.0104 |
| GADD45G | growth arrest and DNA-damage-inducible, gamma | −0.31 | 0.0109 |
| ERBB2 | v-erb-b2 erythroblastic leukemia viral | 0.59 | 0.0122 |
| | oncogene homolog 2 | | |
| KRT16 | keratin 16 | 0.34 | 0.0129 |
| ESR1 | estrogen receptor 1 | 0.63 | 0.0132 |
| FLT3 | fms-related tyrosine kinase 3 | −0.32 | 0.0143 |
| NPTX2 | neuronal pentraxin II | −0.32 | 0.0148 |
| THSD1 | thrombospondin, type I, domain containing 1 | −0.36 | 0.0155 |
| N-PAC | glyoxylate reductase 1 homolog | −0.26 | 0.0162 |
| (GLYR1) | (Arabidopsis) | | |
| TYK2 | tyrosine kinase 2 | −0.33 | 0.0162 |
| ICAM1 | intercellular adhesion molecule 1 | −0.28 | 0.0168 |
| PLEK | Plekstrin | −0.35 | 0.0173 |
| TBC1D3 | TBC1 domain family, member 3 | −0.25 | 0.0179 |
| GPR125 | G protein-coupled receptor 125 | −0.35 | 0.0180 |
| GATA3 | GATA binding protein 3 | 0.28 | 0.0181 |
| TLX3 | T-cell leukemia homeobox 3 | 0.36 | 0.0190 |
| ITGB1 | integrin, beta 1 | −0.21 | 0.0195 |
| CDK2AP1 | cyclin-dependent kinase 2 associated | 0.38 | 0.0205 |
| | protein 1 | | |
| COPB2 | coatomer protein complex, subunit beta 2 | −0.24 | 0.0207 |
| | (beta prime) | | |
| SPINT2 | serine peptidase inhibitor, Kunitz type, 2 | −0.31 | 0.0209 |
| IL16 | interleukin 16 | −0.30 | 0.0218 |
| PAK2 | p21 protein (Cdc42/Rac)-activated kinase 2 | −0.27 | 0.0222 |
| LCP1 | lymphocyte cytosolic protein 1 (L-plastin) | −0.34 | 0.0224 |
| STAT3 | signal transducer and activator of | 0.38 | 0.0224 |
| (pY705) | transcription 3 | | |
| CLU | Clusterin | 0.37 | 0.0226 |
| HIF1A | hypoxia inducible factor 1, alpha subunit | −0.29 | 0.0227 |
| SERPINH1 | serpin peptidase inhibitor, clade H | 0.45 | 0.0227 |
| HOXD13 | homeobox D13 | 0.33 | 0.0229 |
| IGFBP2 | insulin-like growth factor binding protein 2, | −0.33 | 0.0231 |
| | 36 kDa | | |
| CTSE | cathepsin E | −0.31 | 0.0238 |
| STAT3 | signal transducer and activator of | −0.25 | 0.0241 |
| | transcription 3 | | |
| EGFR | epidermal growth factor receptor | −0.27 | 0.0249 |
| APOL1 | apolipoprotein L, 1 | −0.21 | 0.0255 |
| AR | androgen receptor | −0.35 | 0.0267 |
| BCL2 | B-cell CLL/lymphoma 2 | 0.40 | 0.0276 |
| UBE2S | ubiquitin-conjugating enzyme E2S | −0.31 | 0.0286 |
| PRDX3 | peroxiredoxin 3 | −0.32 | 0.0290 |
| PEBP1 | phosphatidylethanolamine binding protein 1 | −0.29 | 0.0293 |
| ABL1 | c-abl oncogene 1, non-receptor tyrosine | −0.27 | 0.0306 |
| | kinase | | |
| BIRC5 | baculoviral IAP repeat containing 5 | −0.21 | 0.0307 |
| MFI2 | antigen p97 (melanoma associated) | −0.27 | 0.0311 |
| STAT6 | signal transducer and activator of | −0.31 | 0.0313 |
| | transcription 6, interleukin-4 induced | | |
| XRCC3 | X-ray repair complementing defective | −0.30 | 0.0321 |
| | repair in CH cells 3 | | |
| CHEK1 | checkpoint kinase 1 | −0.26 | 0.0323 |
| IL1A | interleukin 1, alpha | 0.30 | 0.0326 |
| ZNF331 | zinc finger protein 331 | −0.34 | 0.0326 |
| HDAC2 | histone deacetylase 2 | −0.34 | 0.0330 |
| MLLT10 | myeloid/lymphoid or mixed-lineage | 0.33 | 0.0346 |
| | leukemia, 10 | | |
| ATP8B1 | ATPase, aminophospholipid transporter, | −0.34 | 0.0348 |
| | class I, type 8B, member 1 | | |
| RAB5A | RAB5A, member RAS oncogene family | −0.26 | 0.0361 |
| BRCA1 | breast cancer 1, early onset | −0.27 | 0.0363 |
| DDB2 | damage-specific DNA binding protein 2, | 0.26 | 0.0368 |
| | 48 kDa | | |
| CD20 | membrane-spanning 4-domains, subfamily | 0.21 | 0.0371 |
| (MS4A1) | A, member 1 | | |
| PTPN11 | protein tyrosine phosphatase, non-receptor | −0.14 | 0.0375 |
| | type 11 | | |
| BRAF | v-raf murine sarcoma viral oncogene | 0.27 | 0.0379 |
| | homolog B1 | | |
| FN1 | fibronectin 1 | −0.44 | 0.0382 |
| FCRL5 | Fc receptor-like 5 | −0.27 | 0.0389 |
| TNC | tenascin C | 0.35 | 0.0402 |
| ELP2 | elongator acetyltransferase complex | −0.24 | 0.0405 |
| | subunit 2 | | |
| KIF16B | kinesin family member 16B | −0.34 | 0.0419 |
| PRDX3 | peroxiredoxin 3 | −0.11 | 0.0421 |
| STEAP2 | STEAP family member 2, metalloreductase | 0.29 | 0.0421 |
| NANOG | Nanog homeobox | −0.27 | 0.0422 |
| CD44 | CD44 molecule (Indian blood group) | −0.24 | 0.0446 |
| ALDH1A1 | aldehyde dehydrogenase 1 family, member | −0.24 | 0.0447 |
| | A1 | | |
| TGFB2 | transforming growth factor, beta 2 | −0.36 | 0.0456 |
| ANKRD30A | ankyrin repeat domain 30A | −0.25 | 0.0463 |
| ORM1 | orosomucoid 1 | −0.26 | 0.0466 |
| TMEM173 | transmembrane protein 173 | −0.44 | 0.0469 |
| CHEK1 | checkpoint kinase 1 | −0.32 | 0.0470 |
| KRT14 | keratin 14 | −0.32 | 0.0477 |
| PKM2 | pyruvate kinase, muscle | 0.39 | 0.0478 |
| MUTYH | mutY homolog (E. coli) | −0.24 | 0.0480 |
| CLTC | clathrin, heavy chain (Hc) | −0.26 | 0.0483 |
| NDRG1 | N-myc downstream regulated 1 | 0.45 | 0.0484 |
| DCLK3 | doublecortin-like kinase 3 | −0.27 | 0.0484 |
| PEBP1 | phosphatidylethanolamine binding protein 1 | −0.28 | 0.0487 |
| ESR1 | estrogen receptor 1 | 0.43 | 0.0498 |
| ARMCX1 | armadillo repeat containing, X-linked 1 | −0.24 | 0.0499 |

Antibody microarray interrogation and linear regression analyses were used to identify candidate plasma proteins differentiating pre-diagnostic pancreas cancer plasma from age-matched control samples with statistical significance. The odds ratio (normalized red/green coefficient across all case and control samples) and accompanying p-value for markers are listed in the order of ascending p-values.

As the goal was to identify early disease plasma markers that could be used either individually or in a panel in a non-invasive blood test, we concentrated on the twenty-three up regulated proteins. We cross-referenced our list to that of the pancreaticcancerdatabase.org and Harsha et al. (see, Id.), and found that 15 of these 23 putative markers have been previously associated with pancreatic neoplasms. Eight of these 23 have also been previously reported in the plasma: ERBB2, KRT16, ESR1 (2 antibodies were in the top list), STAT3 (pY705), CLU, SERPINH1, TNC, and PKM. The presence of two distinct antibodies against ESR1 (estrogen receptor 1) in the list of significantly up-regulated pre-diagnostic candidate markers, and a third antibody showing increased levels with a p-value <0.06 (not shown), was intriguing given our pre-diagnostic plasma samples were exclusively from women. Elevated ESR1 has been reported in invasive PDA and estrogen receptor positively is a defining characteristic of mucinous cystic neoplasms (MCN), which are found predominantly in women (Satake et al., Pancreas 33:119-27, 2006).

Therefore, the antibody microarray interrogation of pre-diagnostic human plasma samples successfully identified markers that may allow for the detection pancreatic cancer and precursor lesions using a simple blood test. It is of note that the makers were identified in samples that were collected up to 4-years prior to diagnosis of PDA. Accordingly, these markers have utility for the early detection of pancreatic cancer.

### Example 4

### Cross-Species Biomarker Identification

Comparison of pre-invasive and invasive murine datasets with the pre-diagnostic human data shows that the extracellular matrix marker TNC and the plasma membrane receptor tyrosine kinase ERBB2 are both up-regulated in case plasma samples relative to controls. Plotting the M-values (the normalized red/green ratio) of case and control samples for both pre-diagnostic human and pre-invasive and invasive KPC plasma shows elevated levels of TNC and ERBB2 distinguishing case from control samples with statistical significance (FIGS. 2A, B, D and E).

Increased Erbb2 expression has been demonstrated previously by IHC in pre-invasive and invasive PDA in KPC mice (Hingorani et al., Cancer Cell 7:469-83, 2005), corroborating our findings here. To determine whether tissues also corroborated the elevated plasma TNC levels, we examined tissue collected from cohorts of KPC mice at 2- and 4-months of age, representing pre-invasive and early invasive disease stages, respectively. Array analyses of tissue lysates showed increased TNC as represented by the M-value plots of cases and controls (FIG. 2F), as well as in diagnostic human plasma (FIG. 2G). IHC revealed increasing levels of TNC with progression from PanIN to invasive PDA (FIG. 3). Of note, stromal deposition of TNC was seen in regions surrounding the earliest PanIN-1 lesions (FIG. 3, Panel A). Although others have reported modestly elevated levels of TNC in chronic pancreatitis (Esposito et al., J Pathol, 208:673-85, 2006)—albeit to a lesser extent than in PDA—we did not see appreciable increases in TNC deposition in an experimental mouse model of chronic pancreatitis (FIG. 3, Panels D and H).

Thus, elevated levels of plasma TNC and ERBB2 identified in our cross-species analyses is supported by corresponding increases in primary pancreatic tumor tissue in our study (TNC) and those of others (ERBB2).

### Example 5

### Validation of ESR1, ERBB2 and TNC in Pre-Diagnostic and Diagnostic PDA Plasma

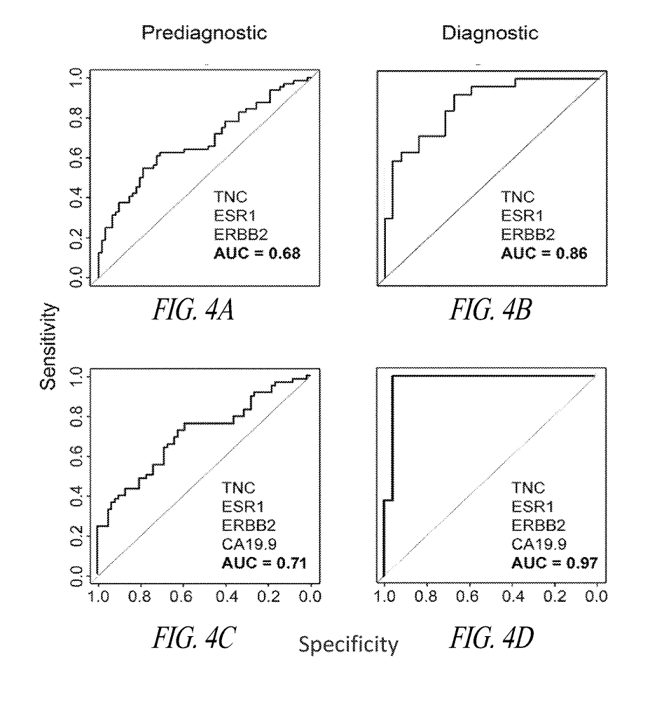

In order to determine the ability of the 3-marker panel to detect pre-diagnostic pancreatic cancer or precursor lesions, a receiver operator characteristic (ROC) curve was calculated for the panel of ERBB2, ESR1 (the two significant antibodies were included) and TNC. The ROC curve yielded an AUC=0.68 (0.58-0.77, 95% CI), with 30% sensitivity at 90% specificity (FIG. 4A). When CA19-9 levels were included, the AUC increased to 0.71 (0.60-0.79, 95% CI, FIG. 4C). Therefore, ROC curve indicates that the markers may have utility in detected pre-diagnostic pancreatic cancer and precursor lesions and may be used for early diagnosis of pancreatic cancer.

In order to validate 3-marker panel, the plasma proteome of diagnostic plasma samples collected through the Center for Accelerated Translation of Pancreas Cancer (CATPAC) was interrogated. CATPAC patient information, including age, stage and diagnosis, and smoking status are included in Table 2. Twenty-four diagnostic plasma samples (13 of which underwent surgical resection) were compared via array to 24 control plasma samples. Unpaired linear regression yielded 243 statistically significant (p-value <0.05) candidates with 133 candidates increased in case versus control plasma (not shown). When evaluating the 23 up-regulated markers from the WHI samples and more specifically the TNC, ERBB2 and ESR1 values within the CATPAC dataset, TNC was again increased with statistical significance (OR=1.86; p-value=0.004) and ERBB2 (OR=1.77; p-value=0.11) and ESR1 (OR=1.62; p-value=0.055), identified in our pre-diagnostic samples, also approached significance in the diagnostic cohort.

As with the pre-diagnostic data, a ROC curve was generated for the diagnostic PDA samples (FIGS. 4B and 4D). The AUC for the 3-marker panel in diagnostic PDA samples was 0.86 (95% CI 0.76-0.96, FIG. 4B). When we include the CA19-9 values measured for our diagnostic samples (Table 4; FIG. 2H), the AUC for the 3-marker panel plus CA19-9 increases to 0.97 (0.92-1.0, 95% CI, FIG. 4D). By comparison, the AUC for CA19-9 alone was 0.84 in these samples, and has been reported as ˜0.78 in other sample sets (Koopmann et al., Clin Cancer Res 12:442-6, 2006).

TABEL 4
The stage at diagnosis and the clinically determined CA19-9 levels for the
diagnostic plasma sample set.

| Stage | CA19-9 (U/ml) |
|---|---|
| III | 1389 |
| III | 1061 |
| IIB | 133 |
| IIB | 401 |
| IA | 63 |
| III | 218,560 |
| IIB | 56 |
| III | 95 |
| IV | 929 |
| IIA | 5990 |
| IIA | 1380 |
| IIB | 42 |
| IIA | 133 |
| IIB | 34 |
| unknown | 20 |
| IB | 43 |
| III | 106 |
| IIA | 4 |
| IV | 4116 |
| III | 108 |
| IIB | 2415 |
| unknown | 2328 |
| III | 2500 |
| IB | 46 |

These studies show that these 3 markers have diagnostic utility, improving upon CA19-9 alone and could be of particular importance for patients that are Lewis negative and cannot express CA19-9 (sialyl-Lewis A). These studies also establish proof of principle that a blood-based assay can identify PDA significantly earlier than current clinical modalities and prior to the onset of symptoms. Furthermore, these markers were able to detect disease in plasma drawn up to 4 years prior to patient diagnosis; an AUC approaching 0.7 for predicting incipient PDA that improves to 0.86 upon diagnosis indicates that these markers can help detect disease sufficiently early to be clinically meaningful.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for detecting the risk of a pancreas hyperproliferative disorder (PHD), comprising identifying the risk of the PHD in a human subject when a test sample from the human subject has at least one biomarker antigen that is elevated compared to a control,
wherein the level of biomarker antigen in the sample is measured by detecting the amount of biomarker antigen in the sample that specifically binds to an antigen binding domain; and
the biomarker antigen comprises at least one of an ERBB2 antigen, an ESR1 antigen, a TNC antigen, or any combination thereof.

2. The method of claim 1, wherein the PHD is pancreatic ductal adenocarcinoma (PDA).

3. The method of claim 1, wherein the PHD is a precursor lesion.

4. The method of claim 3, wherein the precursor lesion is an intraductal papillary mucinous neoplasm (IPMN), mucinous cystic neoplasm (MCN), or pancreatic intraepithelial neoplasia (PanIN).

5. A method for diagnosing a pancreas hyperproliferative disorder (PHD), comprising diagnosing the PHD in a human subject when a test sample from the human subject has at least one biomarker antigen that is elevated compared to a control,
wherein the level of biomarker antigen in the sample is measured by detecting the amount of biomarker antigen in the sample that specifically binds to an antigen binding domain; and
the biomarker antigen comprises at least one of an ERBB2 antigen, an ESR1 antigen, a TNC antigen, or any combination thereof.

6. The method of claim 5, wherein the PHD is pancreatic ductal adenocarcinoma (PDA).

7. The method of claim 5, wherein the PHD is a precursor lesion.

8. The method of claim 7, wherein the precursor lesion is an intraductal papillary mucinous neoplasm (IPMN), mucinous cystic neoplasm (MCN), or pancreatic intraepithelial neoplasia (PanIN).

9. A method of identifying a human subject in need of additional screening for a pancreatic hyperproliferative disorder (PHD), comprising identifying the human subject when a test sample from the human subject has at least one biomarker antigen that is elevated compared to a control,
wherein the level of biomarker antigen in the sample is measured by detecting the amount of biomarker antigen in the sample that specifically binds to an antigen binding domain;
the biomarker antigen comprises at least one of an ERBB2 antigen, an ESR1 antigen, a TNC antigen, or any combination thereof, and
the additional screening comprises at least one of endoscopic ultrasound, computed tomography, magnetic resonance imaging, and biopsy.

10. The method of claim 9, wherein the PHD is pancreatic ductal adenocarcinoma (PDA).

11. The method of claim 9, wherein the PHD is a precursor lesion.

12. The method of claim 11, wherein the precursor lesion is an intraductal papillary mucinous neoplasm (IPMN), mucinous cystic neoplasm (MCN), or pancreatic intraepithelial neoplasia (PanIN).

13. A method of monitoring progression, residual disease, or recurrence of a pancreas hyperproliferative disorder (PHD) in a human subject, comprising detecting the level of at least one biomarker antigen in a sample from a human subject that has received at least one treatment for the PHD and comparing the expression of the biomarker antigen to a control,
wherein the level of biomarker antigen in the sample is measured by detecting the amount of biomarker antigen in the sample that specifically binds to an antigen binding domain; and
the biomarker antigen comprises at least one of an ERBB2 antigen, an ESR1 antigen, a TNC antigen, or any combination thereof.

14. The method of claim 13, wherein the treatment is a surgery, chemotherapy, cytotoxic therapy, immune mediated therapy, targeted therapies, radiation therapy, or a combination thereof.

15. The method of claim 13 or 14, wherein a decrease in at least one of ERBB2, ESR1, and TNC indicates a reduction in tumor burden or a remission.

16. The method of claim 13 or 14, wherein an increase in at least one of ERBB2, ESR1, and TNC indicates an increase in tumor burden or a recurrence of the PHD.

17. The method of claim 13 or 14, wherein the PHD is pancreatic ductal adenocarcinoma (PDA).

18. The method of claim 13 or 14, wherein the PHD is a precursor lesion.

19. The method of claim 17, wherein the precursor lesion is an intraductal papillary mucinous neoplasm (IPMN), mucinous cystic neoplasm (MCN), or pancreatic intraepithelial neoplasia (PanIN).

20. A method of evaluating the efficacy of a pancreas hyperproliferative disorder (PHD) therapy in a human subject comprising administering a PHD therapy to a human subject and determining the efficacy of the therapy by measuring the level of at least one biomarker antigen compared to a control,
wherein the level of biomarker antigen in the sample is measured by detecting the amount of biomarker antigen in the sample that specifically binds to an antigen binding domain; and
the biomarker antigen comprises at least one of an ERBB2 antigen, an ESR1 antigen, a TNC antigen, or any combination thereof.

21. The method of claim 20, wherein the therapy is a surgery, chemotherapy, cytotoxic therapy, immune mediated therapy, targeted therapies, or radiation therapy.

22. The method of claim 20, wherein the PHD is pancreatic ductal adenocarcinoma (PDA).

23. The method of claim 20, wherein the PHD is a precursor lesion.

24. The method of claim 23, wherein the precursor lesion is an intraductal papillary mucinous neoplasm (IPMN), mucinous cystic neoplasm (MCN), or pancreatic intraepithelial neoplasia (PanIN).

25. The method of any one of the preceding claims, wherein the biomarker antigens comprise an ERBB2 antigen, an ESR1 antigen, and a TNC antigen.

26. The method of claim any one of the preceding claims, wherein at least 2 or at least 3 of the biomarker antigens in the sample are elevated.

27. The method of claim 26, wherein at least two of the ERBB2, ESR1, and TNC antigens in the test sample have a level that is elevated compared to the control, wherein the at least two antigens are selected from ERBB2/ESR1, ERBB2/TNC, ESR1/TNC, or ERBB2/ESR1/TNC.

28. The method according to any one of the preceding claims, further comprising detecting the level of a CA19-9 antigen.

29. The method of any one of the preceding claims, wherein the level of expression of the biomarker antigen is at least 2, 3, 4, 5, 6, 7, 8, 9, 10, 15, or 20 fold higher than the control.

30. The method according to any one of the preceding claims, wherein the antigen binding domain is detected with a labeled anti-human immunoglobulin.

31. The method according to claim 30, wherein the anti-human immunoglobulin comprises a fluorescent label.

32. The method according to claim 31, wherein the fluorescent label is a cyanine dye, a coumarin, a rhodamine, a xanthenes, a fluorescein or a sulfonated derivatives thereof, or a fluorescent protein.

33. The method according to claim 30, wherein the anti-human immunoglobulin comprises a chromogenic reporter.

34. The method according to claim 33, wherein the chromogenic reporter comprises a horseradish peroxidase or an alkaline phosphatase.

35. The method according to any one of claims 30-34, wherein the labeled anti-human immunoglobulin is an anti-IgA, anti-IgD, anti-IgE, anti-IgG, or anti-IgM.

36. The method according to any one of claims 30-35, wherein the method comprises a sandwich assay.

37. The method of any of the preceding claims, further comprising the step of performing a endoscopic ultrasound, computed tomography, magnetic resonance imaging, or biopsy on the human subject to confirm the presence of a pancreatic cancer.

38. A method for treating a pancreas hyperproliferative disorder (PHD), comprising administering to a human subject an effective therapeutic regimen for a human subject wherein the PHD is detected in the subject identified when a test sample from the human subject has at least one biomarker antigen that is elevated compared to a control,
wherein the level of biomarker antigen in the sample is measured by detecting the amount of biomarker antigen in the sample that specifically binds to an antigen binding domain; and
the biomarker antigen comprises at least one of an ERBB2 antigen, an ESR1 antigen, a TNC antigen, or any combination thereof.

39. The method of claim 38, wherein the PHD is pancreatic ductal adenocarcinoma (PDA).

40. The method of claim 38, wherein the PHD is a precursor lesion.

41. The method of claim 40, wherein the precursor lesion is an intraductal papillary mucinous neoplasm (IPMN), mucinous cystic neoplasm (MCN), or pancreatic intraepithelial neoplasia (PanIN).

42. The method of any one of claims 38-41 wherein the biomarker antigens comprise an ERBB2 antigen, an ESR1 antigen, and a TNC antigen.

43. The method of any one of claims 38-42, wherein the human subject has a level of a CA19-9 antigen that is elevated compared to a control.

44. The method according to any one of claims 38-43, wherein the therapeutic regimen comprises radiation therapy, chemotherapy, adjunctive therapy, surgery, cytotoxic therapy, immune mediated therapy, targeted therapies, chemoradiotherapy, or any combination thereof.

45. The method of any one of claims 38-44, wherein the PHD is further detected by at least one of an endoscopic ultrasound, computed tomography, magnetic resonance imaging, or biopsy.

46. The method of any one of the preceding claims, wherein the human subject is at high risk for developing a PHD.

47. The method of claim 46, wherein the human subject has a mutation in at least one gene selected from a group comprising BRCA1, BRCA2, P16/INK4A, TP53 (Li-Fraumeni syndrome), palladin (PALLD), FAMMM, Peutz-Jeghers Syndrome, and HNPCC.

48. The method of claim 46, wherein the human subject has at least one first-degree relative that has been diagnosed with pancreatic cancer.

49. The method of claim 46, wherein the human subject has at least two or at least three first degree relatives that have been diagnosed with pancreatic cancer.

50. The method of any one of the preceding claims, wherein the biological sample is blood.

51. The method of any one of the preceding claims, wherein the biological sample is plasma.

52. The method of any one of the preceding claims, wherein specificity for PDA is at least about 90% and sensitivity is at least about 30%.

53. The method of any one of the preceding claims, wherein the level of a further biomarker antigen is measured, wherein the further biomarker antigen is selected from CA19-9 antigen, SEPT5, IL2RA, KRT16, GATA3, TLX3, CDK2AP1, STAT3, CLU, SERPINH1, HOXD13, BCL2, IL1A, MLLT10, DDB2, CD20, BRAF, STEAP2, PKM2, NDRG1, or any combination thereof.
